(19) United States
(12) Patent Application Publication
(10) Pub. No.: US 2012/0225954 A1
(43) Pub. Date: Sep. 6, 2012

(54) METHODS AND COMPOSITIONS FOR THE CLASSIFICATION OF NON-SMALL CELL LUNG CARCINOMA

(75) Inventors: Michael F. Moran, Aurora (CA); Thomas Kislinger, Toronto (CA); Ming-Sound Tsao, Toronto (CA); Yuhong Wei, Toronto (CA); Jiefei Tong, Richmond Hill (CA); Paul Taylor, Waterloo (CA)

(73) Assignees: UNIVERSITY HEALTH NETWORK, Toronto (CA); THE HOSPITAL FOR SICK CHILDREN, Toronto (CA)

(21) Appl. No.: 13/223,862

(22) Filed: Sep. 1, 2011

Related U.S. Application Data
(60) Provisional application No. 61/380,250, filed on Sep. 5, 2010.

(51) Int. Cl. G01N 27/62; A61P 35/00; A61K 35/00; G01N 33/574; C40B 30/10

(52) U.S. Cl. 514/789; 436/501; 506/12; 435/40.52; 435/23

(57) ABSTRACT

The disclosure includes a method of screening for, diagnosing or detecting non-small cell lung carcinoma or an increased likelihood of developing non-small cell lung carcinoma in a subject. The method comprises:
(a) determining the level of at least one biomarker in a test sample from the subject wherein the at least one biomarker is selected from the biomarkers set out in Table 2, 4A, 4B, 6 and/or 7; and
(b) comparing the level of the at least one biomarker in the test sample with a control;
   wherein detecting a difference in the level of the at least one biomarker in the test sample compared to the control is indicative of whether the subject has or does not have non-small cell lung carcinoma or an increased likelihood of developing non-small cell lung carcinoma.

### CROSS REFERENCE TO RELATED APPLICATION

This application claims the benefit of 35 USC 119 based on the priority of copending U.S. Provisional Application No. 61/380,250 filed Sep. 5, 2010, which is herein incorporated by reference.

### SEQUENCE LISTING

A computer readable form of the Sequence Listing “10723-380.txt” (3283 bytes), submitted via EFS-WEB and created on Sep. 1, 2011 is herein incorporated by reference.

### FIELD

The application relates to lung cancer and particularly to methods, compositions and kits for classifying subjects with the adenocarcinoma (ADC) subtype or squamous cell carcinoma (SCC) subtype of non-small cell lung carcinoma (NSCLC) according to protein signatures.

### INTRODUCTION

Lung cancer is the most common cause of death from cancer for both men and women, with a current worldwide mortality rate in excess of one million per year. Non-small cell lung carcinoma (NSCLC) is histologically heterogeneous, with adenocarcinoma (ADC), squamous cell carcinoma (SCC), and large cell carcinoma (LC) being the major subtypes ¹. Combined, these subtypes account for approximately 85% of lung cancers. In clinical practice, these subtypes have been treated similarly until recently, when new therapies (e.g. premetrexed) have shown differential responses in NSCLC subtypes². However, despite improvements in surgical and chemotherapeutic treatments, and the development of drugs targeting the epidermal growth factor receptor (EGFR), which is a target in a subset of NSCLC, the 5-year survival rate associated with these cancers is poor, at approximately 15%. There is considerable variability in the molecular features between and within each of these NSCLC subtypes (e.g. EGFR expression level and mutational status), suggesting that additional stratification of tumors may facilitate more effective, tumor-specific treatments ³.

The analysis of EGFR and various keratins by methods with limited dynamic range such as immunohistochemistry (IHC) are common practices in oncologic pathology. EGFR levels by IHC have not proven to be predictive of response to EGFR-directed drugs, despite initial studies suggesting that patients whose tumors demonstrate low expression have low response rates ⁴.

The keratins are relatively abundant proteins (i.e. expressed at high level), and are the major structural component of the intermediate filament-based epithelial barrier in tissue ⁵. Keratin expression is stable during tumorigenesis, and the keratin expression pattern may signify tumor origins and types⁵. Indeed, since keratins exhibit characteristic expression patterns in human tumors, several of them (notably K5, K7, K8/K18, K19 and K20) have great importance in immunohistochemical tumor diagnosis of carcinomas, in particular of unclear metastases and in precise classification and subtyping⁵. However, it has been found that there is a limited differential expression of distinctive keratin filaments between squamous cell carcinomas and adenocarcinomas²⁷. Apparently, squamous cell carcinomas that originate from columnar epithelium by squamous metaplasia gain the keratins of squamous cells but retain the keratins of columnar epithelial cells²⁷.

While some keratins have been detected in blood and monitored as biomarkers (e.g. CYFRA 21-1 fragment of KRT19 ⁶), only a subset of the 54 human keratin proteins have been developed into clinically useful diagnostic biomarkers to-date. There remains an unmet need to develop sensitive and more quantitative methods to identify and quantify comprehensive sets of diagnostic biomarkers including drug targets such as the EGFR and their associated signaling network components, and protein classes such as the keratins whose function is involved in the epithelial tissue and tumor phenotypes, and which may inform of tumor subtypes.

Mass spectrometry (MS) has emerged as a powerful technology for proteomic analysis of tumors, and represents a promising approach to stratify tumors according to their protein profiles, and for drug target and biomarker discovery ⁷. These methods have been extensively reviewed, and applied largely to study tumor-derived cell lines grown either in two-dimensional cultures or as xenograft tumors in immuno deficient mice. However, in either growth context, such established cell lines are mostly not representative of the more diversified or heterogeneous tumors in human cancers ⁸. Another issue associated with MS analysis of human-murine xenograft systems is the recognition and assignment of human versus murine proteins, which share a large degree of sequence homology. Methods to recognize and quantify human tumor proteomes, and to generate tissue models that faithfully retain or recapitulate their protein profiles are required.

These findings illustrate the potential to develop a comprehensive MS-based platform in oncologic pathology for better classification and potentially treatment of NSCLC patients.

### SUMMARY

Non-small cell lung carcinoma (NSCLC) accounts for approximately 80% of lung cancer. The most prevalent subtypes of NSCLC are adenocarcinoma (ADC) and squamous cell carcinoma (SCC), which combined account for approximately 90% of NSCLCs. Ten resected NSCLC patient tumors (5 ADC and 5 SCC) were directly introduced into severely immune deficient (NOD-SCID) mice, and the resulting xenograft tumors analyzed by standard histology and immunohistochemistry (IHC), and by proteomics profiling. Mass spectrometry (MS) methods involving 1- and 2-dimensional LC-MS/MS, and multiplexed selective reaction monitoring (SRM, or MRM) were applied to identify and quantify the xenograft proteomes. Hierarchical clustering of protein profiles distinguished between the ADC and SCC subtypes. As an example, the differential expression of 178 proteins, including a comprehensive panel of intermediate filament keratin proteins was found to constitute a distinctive proteomic signature associated with the NSCLC subtypes and subsets of proteins were found to be highly expressed in ADC or SCC. Epidermal growth factor receptor (EGFR) was expressed in ADC and SCC xenografts, and EGFR network activation was assessed by phosphotyrosine profiling by western blot analysis and SRM measurement of EGFR levels, and mutation analysis. A multiplexed SRM/MRM method provided relative quantification of several keratin proteins, EGFR and plakophilin-1 in single LC-MS/MS runs. Protein quantifications by SRM and MS/MS spectral counting were consistent with, and validated by orthogonal methods including IHC and Western immunoblotting.

Accordingly, an aspect includes a method of screening for, diagnosing or detecting non-small cell lung carcinoma or an increased likelihood of developing non-small cell lung carcinoma in a subject. The method comprises:
(a) determining the level of at least one biomarker in a test sample from the subject wherein the at least one biomarker is selected from the biomarkers set out in Table 2, 4A, 4B, 6 and/or 7; and
(b) comparing the level of the at least one biomarker in the test sample with a control;
   wherein detecting a difference in the level of the at least one biomarker in the test sample compared to the control is indicative of whether the subject has or does not have non-small cell lung carcinoma or an increased likelihood of developing non-small cell lung carcinoma.

Another aspect includes a method of screening for, diagnosing or detecting non-small cell lung carcinoma or an increased likelihood of developing non-small cell lung carcinoma in a subject comprising:
(a) determining a level of at least one biomarker associated with non-small cell lung carcinoma in a test sample from the subject, the at least one biomarker selected from the biomarkers set out in Table 2, 4A, 4B, 6 and/or 7; and
(b) comparing the level of the at least one biomarker in the test sample with a control;
   wherein detecting a difference in a level of the at least one biomarker in the test sample compared to the control is indicative of whether the subject has or does not have non-small cell lung carcinoma or an increased likelihood of developing non-small cell lung carcinoma.

A further aspect includes a method of differentiating between non-small cell lung carcinoma of the adenocarcinoma subtype and non-small cell lung carcinoma of the squamous cell carcinoma subtype in a subject, or detecting an increased likelihood of developing non-small cell lung carcinoma of the adenocarcinoma subtype or non-small cell lung carcinoma of the squamous cell carcinoma subtype in a subject comprising:
(a) determining a level of at least one biomarker associated with non-small cell lung carcinoma of the adenocarcinoma subtype or non-small cell lung carcinoma of the squamous cell subtype in a test sample from the subject wherein the at least one biomarker is selected from the biomarkers set out in Table 2, 4A, 4B, 6 and/or 7; and
(b) comparing the level of the at least one biomarker in the test sample with a control;
   wherein detecting a difference or similarity in a level of the at least one biomarker in the test sample compared to the control is indicative of whether the subject has non-small cell lung carcinoma of the adenocarcinoma subtype or non-small cell lung carcinoma of the squamous cell carcinoma subtype, or an increased likelihood of developing non-small cell lung carcinoma of the adenocarcinoma subtype or non-small cell lung carcinoma of the squamous cell carcinoma subtype.

Another aspect includes A method of screening for, diagnosing or detecting non-small cell lung carcinoma of adenocarcinoma subtype or an increased likelihood of developing non-small cell lung carcinoma of the adenocarcinoma subtype in a subject comprising:
(a) determining a level of at least one biomarker associated with non-small cell lung carcinoma of the adenocarcinoma subtype in a test sample from the subject wherein the at least one biomarker is selected from the biomarkers set out in Table 2 or Table 4A; and
(b) comparing the level of the at least one biomarker in the test sample with a control;
   wherein detecting a difference or similarity in the level of the at least one biomarker in the test sample compared to the control is indicative of the subject has or does not have non-small cell lung carcinoma of adenocarcinoma subtype or an increased likelihood of developing non-small cell lung carcinoma of adenocarcinoma subtype.

Furthermore, an aspect includes a method of screening for, diagnosing or detecting non-small cell lung carcinoma of squamous cell carcinoma subtype or an increased likelihood of developing non-small cell lung carcinoma of squamous cell carcinoma subtype in a subject comprising:
(a) determining a level of at least one biomarker associated with non-small cell lung carcinoma of squamous cell carcinoma subtype in a test sample from the subject wherein the at least one biomarker is selected from the biomarkers set out in Table 2 or Table 4B; and
(b) comparing the level of the at least one biomarker in the test sample with a control;
   wherein detecting a difference or similarity in the level of the at least one biomarker in the test sample compared to the control is indicative of whether the subject has or does not have non-small cell lung carcinoma of squamous cell carcinoma subtype or an increased likelihood of developing non-small cell lung carcinoma of squamous cell carcinoma subtype.

Additionally, another aspect includes a SRM/MRM method for quantifying a level of at least one biomarker associated with non-small cell lung carcinoma in a sample, the method comprising the steps of:
a) isotope labeling a peptide fragment of the at least one biomarker wherein the at least one biomarker is selected from the biomarkers set out in Table 2, 4A, 4B, 6 and/or 7; and
b) evaluating the biomarker level using SRM/MRM mass spectrometry.

Moreover, a further aspect includes A kit for measuring a level of at least one biomarker associated with non-small cell lung cancer or a subtype thereof in a sample, the at least one biomarker selected from the biomarkers set out in Table 2, 4A, 4B, 6 and/or 7, comprising:
a) a biomarker specific reagent, labeling isotope and/or a peptidase such as trypsin;
b) a kit control, optionally a peptide fragment of a biomarker;
c) optionally an array slide; and
d) optionally instructions for use.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be described in relation to the drawings in which:

FIG. 1 illustrates the parallel collection of pathology and proteomics data sets, which were compared, and subjected to further validation by immunohistochemistry (IHC) and Western blotting, and quantification by multiplexed SRM-MS (also known as MRM).

FIG. 2 illustrates the hematoxylin/eosin stain of primary NSCLC xenografts including 5 adenocarcinoma (ADC) models, and 5 squamous cell carcinoma (SCC) models.

FIG. 3 illustrates the recognition of ADC and SCC subtypes of NSCLC by 1D LC-MS/MS protein profiling. Dendogram produced by hierarchical clustering of proteins measured by 1D LC-MS/MS and having spectra and sample incidence (541 proteins).

FIG. 4 illustrates the cluster analysis of human proteins in NSCLC. Hierarchical clustering of 2D LC-MS/MS spectra of human proteins resolved ADC (lighter bar) and SCC (darker bar) subtypes. Included were the 1303 proteins with spectral counts and sample incidence ≧2.

FIG. 5 illustrates the comparison of KRT7 by immunohistochemistry and proteomics in NSCLC xenografts. A, KRT7 immunohistochemistry. B, Histograms presenting relative KRT7 expression measured by SRM (see Table 6 for peptide transitions), normalized to SRM-measured actin, in xenograft samples (upper two charts, n=2, error bars shown range), and by spectral counting (lower chart, n=3±SD).

FIG. 6 illustrates the immunohistochemistry and SRM analysis of KRT5 and KRT19 in NSCLC xenografts. A, KRT5 immunohistochemistry. B, SRM analysis of KRT5 peptides, as listed in Table 6. C, KRT19 immunohistochemistry. D, SRM analysis of KRT19 peptides, as listed in Table 6.

FIG. 7 illustrates the immunohistochemistry and SRM analysis of KRT14 in NSCLC xenografts. A, KRT14 immunohistochemistry. B, SRM analysis of a KRT14 peptide, as listed in Table 6.

FIG. 8 illustrates the SRM measurements of KRT15, KRT13, and plakophilin-1 in NSCLC xenografts. See Table 6 for SRM transitions and associated peptides.

FIG. 9 illustrates the analysis of EGFR expression and activation in NSCLC xenografts. A, EGFR protein measured by spectral counting (n=3, ±SD). B,C, Anti-EGFR western blotting (n=3, ±SD). Receptor phosphorylation at Y1068 was imaged by Western analysis as indicated (pEGFR), and compared with total cellular anti-phosphotyrosine (pTyr) staining. Arrows indicate migration of EGFR proteins. D, SRM measurement of two EGFR peptides, as listed in Table 6 (n=2, error bars denote range).

FIG. 10 illustrates the immunohistochemistry of EGFR in 10 NSCLC xenografts. EGFR immunohistochemistry of representative sections in the indicated ten NSCLC xenograft tumors.

FIG. 11 illustrates the Venn Diagram of Technical and Biological Reproducibility in 1D LC-MS/MS.

Table 1 displays NSCLC tumor xenograft histopathology and molecular features.

Table 2 lists highly differentially expressed proteins in ADC and SCC xenografts

Table 3 displays LC-MS/MS protein profiling.

Table 4 lists proteins highly differentially expressed in NSCLC.

Table 5 lists keratin signatures in NSCLC.

Table 6 lists transitions measured by multiplexed SRM/MDM.

Table 7 lists a set of biomarkers of the disclosure.

### DESCRIPTION OF VARIOUS EMBODIMENTS

### I. Definitions

The term “difference in the level” as used herein refers to an increase or decrease in the level, or quantity, of a biomarker associated with non-small cell lung carcinoma or a subtype thereof, in a test sample that is measurable, compared to a suitable control and/or reference. For example the difference can be a difference in the steady-state level of a gene transcript, including for example a difference resulting from a difference in the level of transcription and/or translation and/or degradation. The difference in the level is optionally a level statistically associated with a particular group or outcome, for example, a group having non-small cell lung carcinoma or not having non-small cell lung carcinoma. The difference in the level can refer to an increase or decrease in a measurable polypeptide, or fragment thereof, level of a given biomarker as measured by the amount of steady state level of and/or expressed polypeptide or fragment thereof in a test sample as compared with the measurable expression level of a given biomarker or fragment thereof in a control, population of control samples and/or previously taken or reference sample. In another example, the difference in the level can refer to an increase or decrease in the measurable polynucleotide (e.g. nucleic acid transcript) level of a given biomarker as measured by the amount of transcript e.g. biomarker mRNA or cDNA. For example, in methods relating to screening for, diagnosing or detecting non-small cell lung carcinoma, a difference in the level can refer to an increase in the level of a biomarker compared to a suitable control, wherein the control for example corresponds to a biomarker level in a subject without non-small cell lung carcinoma. In methods relating to monitoring therapeutic response, a difference in the level can refer to a decrease or increase in the level of the biomarker in the subsequent sample compared to a reference sample, wherein depending on the particular biomarker an increase is indicative of negative therapeutic response and/or a decrease is indicative of a positive therapeutic response. For example, a difference in a level of biomarker level is detected if a ratio of the level in a test sample as compared with a control is greater than or less than 1.0 and/or if the ratio of the level in a reference sample as compared with a subsequent sample is greater than or less than 1.0. For example, the ratio can be greater than 1.0, 1.2, 1.5, 1.7, 2, 3, 3, 5, 10, 12, 15, 20 or more, or less than 1, 0.8, 0.6, 0.4, 0.2, 0.1, 0.05, 0.001 or less. The difference in the level when compared to a population average can for example be expressed using p-value. For instance, when using p-value, a biomarker is identified as having a difference in level between a first and second population when the p-value is less than 0.1, such as less than 0.05, 0.01, 0.005, and/or less than 0.001.

The term “biomarker associated with non-small cell lung cancer” as used herein refers to a gene listed in Tables 2, 4A, 4B, 6 and/or 7 or an expression product (e.g. polypeptide or nucleic acid transcript) of such a gene or a fragment thereof such as a peptide, e.g. generated by tryptic digest that is associated with, and an indicator of, pathogenic processes relating to non-small cell lung carcinoma or a subtype thereof. For example, the biomarker can refer to a gene product, such as a polypeptide or fragment thereof, that is differentially detectable for example differentially expressed, in subjects with non-small cell lung carcinoma or a subtype thereof as compared to subjects without non-small cell lung carcinoma or the particular subtype. Similarly, the term “biomarker associated adenocarcinoma” as used herein refers to a gene set out in Tables 2, 4a, and 7 or expression product (e.g. polypeptide or nucleic acid transcript) of such a gene or a fragment that is associated with non-small cell lung cancer of the adenocarcinoma subtype; and, the term “biomarker associated squamous cell carcinoma” as used herein refers to a gene set out in Tables 2, 4B, and 7 or expression product (e.g. polypeptide or nucleic acid transcript) of such a gene or a fragment that is associated with non-small cell lung cancer of the squamous cell carcinoma subtype. The “biomarkers of the disclosure” refer to the biomarkers as set out in Tables 2, 4A, 4B, 6 and/or 7.

The phrase “biomarker polypeptide”, “polypeptide biomarker” or “polypeptide product of a biomarker” refers to a proteinaceous biomarker gene product or fragment thereof. For example, a biomarker polypeptide refers to a Table 2, 4A, 4B, 6 and/or 7 polypeptide biomarker or fragment thereof that is for example, increased in samples from subjects with non-small cell lung carcinoma or a subtype thereof.

The term “biomarker fragment” refers to a polypeptide or polynucleotide that is, in terms of amino acids or nucleotides, less in number than the full length biomarker. For example, a fragment can be at least 7, 10, 20, 30 of any number in between or a corresponding number of nucleotides.

The term “control” as used herein refers to a sample, and/or a biomarker level, numerical value and/or range (e.g. control range) corresponding to the biomarker level in such a sample, taken from or associated with a subject or a population of subjects (e.g. control subjects) who are known as not having non-small cell lung carcinoma or who are known as not having a particular subtype of non-small cell lung carcinoma. For example, in methods for determining if a subject has non-small cell lung carcinoma of the adenocarcinoma subtype, the control can be a sample, and/or a biomarker level, numerical value and/or range (e.g. control range) corresponding to the biomarker level in such a sample, taken from or associated with a subject or a population of subjects (e.g. control subjects) who are known as not having non-small cell lung carcinoma or as who are known as having non-small cell lung carcinoma of the squamous cell carcinoma subtype. Also, for example in methods for determining if a subject has non-small cell lung carcinoma of the squamous cell carcinoma subtype, the control as used herein can be a sample, and/or a biomarker level, numerical value and/or range (e.g. control range) corresponding to the biomarker level in such a sample, taken from or associated with a subject or a population of subjects (e.g. control subjects) who are known as not having non-small cell lung carcinoma or as having non-small cell lung carcinoma of the adenocarcinoma subtype.

Where the control is a numerical value or range, the numerical value or range is a predetermined value or range that corresponds to a level of the biomarker or range of levels of the biomarker in a group of subjects known as not having non-small cell lung carcinoma or subtype thereof (e.g. threshold or cutoff level; or control range). For example, the control can be a cut-off or threshold level, above or below which (depending on the biomarker and subtype) which a subject is identified as having non-small cell lung cancer or a particular subtype thereof. For example, a test subject that has an increased level of a biomarker above a cut-off or threshold level is indicated to have or is more likely to have non-small cell lung carcinoma of a particular subtype.

The term “positive control” as used herein refers to a sample and/or biomarker level or numerical value corresponding to the biomarker level in a sample from a subject or a population of subjects (e.g. positive control subjects) who are known as having non-small cell lung carcinoma, for example a particular subtype of non-small cell carcinoma. For example, in methods for determining if a subject has non-small cell lung carcinoma of the adenocarcinoma subtype, the “positive control” can be a sample and/or biomarker level or numerical value corresponding to the biomarker level in a sample from a subject or a population of subjects (e.g. positive control subjects) who are known as having non-small cell lung carcinoma of the adenocarcinoma subtype. Similarly, in methods for determining if a subject has non-small cell lung carcinoma of the squamous cell carcinoma subtype, the “positive control” can be a sample and/or biomarker level or numerical value corresponding to the biomarker level in a sample from a subject or a population of subjects (e.g. positive control subjects) who are known as having non-small cell lung carcinoma of the squamous cell carcinoma subtype.

The term “similar” in the context of a biomarker level as used herein refers to a subject biomarker level that falls within the range of levels associated with a particular class for example associated with non-small cell lung cancer of adenocarcinoma subtype or associated with non-small cell lung cancer of squamous cell carcinoma subtype. Accordingly, “detecting a similarity” refers to detecting a biomarker level that fall within the range of levels associated with a particular class. In the context of a reference profile, “similar” refers to a reference profile associated with a non-small cell lung cancer subtype such as adenocarcinoma subtype or squamous cell carcinoma subtype that shows a number of identities and/or degree of changes with the subject expression profile.

The term “most similar” in the context of a reference profile refers to a reference profile that is associated with a non-small cell lung cancer subtype such as adenocarcinoma subtype or squamous cell carcinoma subtype that shows the greatest number of identities and/or degree of changes with the subject expression profile.

The term “expression profile” as used herein refers to, for a plurality of biomarkers that are associated with non-small cell lung carcinoma or a subtype thereof, biomarker steady state and/or transcript expression levels in a sample from a subject that is for example, useful for diagnosing non-small cell lung cancer, for example of the adenocarcinoma or squamous cell carcinoma cell type. For example, an expression profile can comprise the quantitated relative levels of at least 2 or more biomarkers listed in Table 2, 4A, 4B, 6 and/or 7, and the levels or pattern of biomarker expression can be compared to one or more reference profiles, for example a reference profile associated with non-small cell lung carcinoma such as non-small cell lung carcinoma of the adenocarcinoma subtype or non-small cell lung carcinoma of the squamous cell carcinoma subtype. An expression profile can for example be detected by microarray analysis, RT-PCR and/or methods that measure a biomarker expression product such as flow cytometry and Western blot.

The term “sequence identity” as used herein refers to the percentage of sequence identity between two or more polypeptide sequences or two or more nucleic acid sequences that have identity or a percent identity for example about 70% identity, 80% identity, 90% identity, 95% identity, 98% identity, 99% identity or higher identity or a specified region. To determine the percent identity of two or more amino acid sequences or of two or more nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted.

The term “specifically binds” as used herein refers to a binding reaction that is determinative of the presence of the biomarker (e.g. polypeptide or nucleic acid) often in a heterogeneous population of macromolecules. For example, when the biomarker specific reagent is an antibody, specifically binds refers to the specified antibody binding with greater affinity to the cognate antigenic determinant than to another antigenic determinant, for example binds with at least 2, at least 3, at least 5, or at least 10 times greater specificity; and when a probe, specifically binds refers to the specified probe under hybridization conditions binds to a particular gene sequence at least 1.5, at least 2 at least 3, or at least 5 times background.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The term “polynucleotide”, “nucleic acid” and/or “oligonucleotide” as used herein refers to a sequence of nucleotide or nucleoside monomers consisting of naturally occurring bases, sugars, and intersugar (backbone) linkages, and is intended to include DNA and RNA which can be either double stranded or single stranded, represent the sense or antisense strand.

The term “primer” as used herein refers to a polynucleotide, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

The term “probe” as used herein refers to a nucleic acid sequence that will hybridize to a nucleic acid target sequence. In one example, the probe hybridizes to a biomarker RNA or a nucleic acid sequence complementary to the biomarker RNA. The length of probe depends for example, on the hybridization conditions and the sequences of the probe and nucleic acid target sequence. The probe can be for example, at least 15, 20, 25, 50, 75, 100, 150, 200, 250, 400, 500 or more nucleotides in length.

A person skilled in the art would recognize that “all or part of” of a particular probe or primer can be used as long as the portion is sufficient for example in the case a probe, to specifically hybridize to the intended target and in the case of a primer, sufficient to prime amplification of the intended template.

The term “EGFR directed drug” as used herein refers to drugs that specifically bind with high affinity to the epidermal growth factor receptor (EGFR) on the cell surface or to the intracellular catalytic region in order to regulate the intrinsic protein-tyrosine kinase activity of the receptor. The tyrosine kinase activity initiates signal transduction cascades that results in a variety of biochemical changes in the cell such as increased aerobic glycolysis, changes in cell-cell and cell-matrix interactions and motility, changes in the expression of certain genes that ultimately lead to DNA synthesis and cell proliferation. EGFR genetic mutations that lead to increased expression or activity of the EGFR protein have been associated with a number of cancers, including lung cancer.

The term “kit control” as used herein means a suitable assay control useful when determining a level of a biomarker associated with non-small cell lung cancer. For example, when the kit is for a MRM/SRM method, the kit control is optionally a peptide fragment of a biomarker polypeptide that can for example be used to prepare a standard curve As an alternative example, where the kit is for detecting polypeptide levels by immunohistochemical methods, the kit control can comprise an antibody control, useful for example for detecting non-specific binding and/or for standardizing the amount of protein in the sample.

The term “biomarker specific reagent” as used herein refers to a reagent that is a highly sensitive and specific biomarker reagent used with standard immunohistochemistry (ICC) and immunohistochemistry (IHC) techniques to detect the level of a biomarker associated with non-small cell lung cancer.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

Antibodies may be monospecific, bispecific, trispecific or of greater multispecificity. Multispecific antibodies may immunospecifically bind to different epitopes of a NADPH oxidase polypeptide and/or or a solid support material. Antibodies may be from any animal origin including birds and mammals (e.g., human, murine, donkey, sheep, rabbit, goat, guinea pig, camel, horse, or chicken).

Antibodies may be prepared using methods known to those skilled in the art. Isolated native or recombinant polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies.

In aspects, the antibody is a purified or isolated antibody. By “purified” or “isolated” is meant that a given antibody or fragment thereof, whether one that has been removed from nature (isolated from blood serum) or synthesized (produced by recombinant means), has been increased in purity, wherein “purity” is a relative term, not “absolute purity.” In particular aspects, a purified antibody is 60% free, preferably at least 75% free, and more preferably at least 90% free from other components with which it is naturally associated or associated following synthesis.

The term “control level” refers to a biomarker level in a control sample or a numerical value corresponding to such a sample. Control level can also refer to for example a threshold, cut-off or baseline level of a biomarker in subjects without non-small cell lung carcinoma or without a particular sub-type, where levels above/below which depending on the particular marker are associated with the presence of non-small cell lung carcinoma or a particular sub-type.

Similarly the term “positive control level” refers to a biomarker level in or corresponding to a positive control sample, for example associated with a subtype of non-small cell lung carcinoma. Positive control level can refer to a threshold, cut-off or baseline level of a biomarker in subjects with non-small cell lung carcinoma or a subtype thereof that is useful for comparing to a subject biomarker level. The positive control can for example be a level of at least one biomarker associated with non-small cell lung cancer or a subtype thereof, or a reference profile comprising levels of a plurality of markers.

The term “sample” as used herein refers to any biological fluid, cell or tissue sample from a subject including a test sample from a test subject e.g. a subject whose lung cancer status is being tested, and a control sample from a control subject e.g. a subject with lung cancer status is known. For example, the sample can comprise lung tissue, tumour biopsy, ascitic fluid, sputum, and/or bodily secretions. The sample for example can comprise formalin fixed and/or paraffin embedded tissue, a frozen tissue or fresh tissue. The sample can be used directly as obtained from the source or following a pretreatment to modify the character of the sample.

The term an “increased likelihood of developing”, as used herein is used to mean that a test subject with increased levels of a biomarker in Table 2, 4A, 4B, 6 and/or 7 has an increased chance of developing non-small cell lung carcinoma, or a subtype thereof, having recurrence or relapse or poorer survival relative to a control subject (e.g. a subject with control levels of a Table 2, 4A, 4B, 6 and/or 7 biomarker). The increased risk for example may be relative or absolute and may be expressed qualitatively or quantitatively. For example, an increased risk may be expressed as simply determining the test subject's expression level for a given biomarker and placing the test subject in an “increased risk” category, based upon previous population studies. Alternatively, a numerical expression of the test subject's increased risk may be determined based upon biomarker level analysis. As used herein, examples of expressions of an increased risk include but are not limited to, odds, probability, odds ratio, p-values, attributable risk, relative frequency, positive predictive value, negative predictive value, and relative risk.

The term “level” as used herein refers to a quantity of biomarker that is detectable or measurable in a sample and/or control. The quantity is for example a quantity of polypeptide, the quantity of nucleic acid e.g. biomarker transcript, or the quantity of a fragment. The level can alternatively include combinations thereof.

The term “determining a level” as used in reference to a biomarker means the application of a method to a sample, for example a sample of the subject and/or a control sample, for ascertaining quantitatively, semi-quantitatively or qualitatively the amount of a biomarker, for example the amount of biomarker polypeptide or mRNA. For example, a level of a biomarker can be determined by a number of methods including for example mass spectrometric methods, including for example MS, MS/MS, LC-MS/MS, SRM etc where a peptide of a biomarker is labeled and the amount of labeled biomarker peptide is ascertained, immunoassays including for example immunohistochemistry, ELISA, immunoprecipation and the like, where a biomarker detection agent such as an antibody for example, a labeled antibody specifically binds the biomarker and permits for example relative or absolute ascertaining of the amount of polypeptide biomarker, hybridazation and PCR protocols where a probe or primer or primer set are used to ascertain the amount of nucleic acid biomarker.

The term “MS” refers to mass spectrometry.

The term “MS/MS” refers to tandem mass spectrometry.

The term “1D LC-MS/MS” refers to 1-dimensional liquid chromatography tandem mass spectrometry using for example a LTQ-Orbitrap XL apparatus.

The term “2D LC-MS/MS” refers to 2-dimensional liquid chromatography tandem mass spectrometry.

The term “SRM” refers to selective reaction monitoring which is a mass spectrometry approach to the quantitative detection of selected proteins. The assay can for example be multiplexed (e.g. MRM).

The term “non-small cell lung carcinoma” or NSCLC as used herein refers to all lung cancers that are not small cell lung cancer and includes several sub-types including but not limited to large cell carcinoma, squamous cell carcinoma and adenocarcinoma. All stages and metastasis are included. Accounting for 25% of lung cancers, squamous cell carcinoma usually starts near a central bronchus. A hollow cavity and associated necrosis are commonly found at the center of the tumor. Well-differentiated squamous cell cancers often grow more slowly than other cancer types. Adenocarcinoma accounts for 40% of non-small cell lung cancers. It usually originates in peripheral lung tissue. Most cases of adenocarcinoma are associated with smoking; however, among people who have never smoked, adenocarcinoma is the most common form of lung cancer.

The term “proteome” as used herein refers to a set of polypeptides, detectable in a sample type, such as a biopsy comprising a lung cell, and/or refers to a set of polypeptides detectable and/or quantified in a cell and/or tumour, for example non-small cell lung carcinoma or a subtype thereof, optionally expressed at a given time and/or under defined conditions.

The term “reference profile” as used herein refers to a suitable comparison profile, for example a polypeptide or nucleic acid reference profile that comprises the level of two or more biomarkers of the disclosure in a sample corresponding to a subject that has or does not have a non-small cell lung carcinoma, or particular subtype thereof. For example, in methods involving determining for example if a subject has non-small cell lung carcinoma of the adenocarcinoma subtype, the “reference profile” can be a polypeptide profile corresponding to a subject that does not have non-small cell lung cancer or who has non-small cell lung carcinoma of the adenocarcinoma subtype. Similarly, in methods involving determining for example if a subject has non-small cell lung carcinoma of the squamous cell carcinoma subtype, the “reference profile” can be a polypeptide reference profile corresponding to a subject that does not have non-small cell lung carcinoma or has non-small cell lung carcinoma of the squamous cell carcinoma subtype. The reference profile is an expression signature (e.g. polypeptide or nucleic acid gene expression levels and/or pattern) of a plurality of genes (e.g. at least 2 genes, for example 5 genes), associated with non-small cell lung cancer or a subtype thereof. The reference profile is identified using one or more samples comprising non-small cell lung cancer cells wherein the expression is similar between related samples defining a subtype class and is different to unrelated samples defining a different subtype class such that the reference expression profile is associated with a particular cancer subtype. The reference expression profile is accordingly a reference profile or reference signature of the expression of five or more genes listed in Table 2, 4A, 4B, 6 and/or 7, to which the expression levels of the corresponding genes in a test sample are compared in methods for example for determining non-small cell lung cancer subtype.

The phrase “screening for, diagnosing or detecting non-small cell lung carcinoma or an increased likelihood of developing non-small cell lung” refers to a method or process of determining if a subject has or does not have non-small cell lung carcinoma, or has or does not have an increased risk of developing non-small cell lung carcinoma. For example, detection of altered levels of a Table 2, 4A, 4B, 6 and/or 7 biomarker compared to control is indicative that the subject has non-small cell lung carcinoma or an increased risk of developing non-small cell lung carcinoma.

The phrase “screening for, diagnosing or detecting non-small cell lung carcinoma of the adenocarcinoma subtype or an increased likelihood of developing non-small cell lung of the adenocarcinoma subtype” refers to a method or process of determining if a subject has or does not have non-small cell lung carcinoma of the adenocarcinoma subtype, or has or does not have an increased risk of developing non-small cell lung carcinoma of the adenocarcinoma subtype. For example, detection of altered levels of a Table 2 or 4A biomarker compared to control is indicative that the subject has non-small cell lung carcinoma of the adenocarcinoma subtype or an increased risk of developing non-small cell lung carcinoma of the adenocarcinoma subtype.

The phrase “screening for, diagnosing or detecting non-small cell lung carcinoma of the squamous cell carcinoma subtype or an increased likelihood of developing non-small cell lung of the squamous cell carcinoma subtype” refers to a method or process of determining if a subject has or does not have non-small cell lung carcinoma of the squamous cell carcinoma subtype, or has or does not have an increased risk of developing non-small cell lung carcinoma of the squamous cell carcinoma subtype. For example, detection of altered levels of a Table 2 or 4B biomarker compared to control is indicative that the subject has non-small cell lung carcinoma of the squamous cell carcinoma subtype or an increased risk of developing non-small cell lung carcinoma of the squamous cell carcinoma subtype.

The term “subject” as used herein refers to any member of the animal kingdom, preferably a human being.

The phrase “therapy or treatment” as used herein, refers to an approach aimed at obtaining beneficial or desired results, including clinical results and includes medical procedures and applications including for example chemotherapy, pharmaceutical interventions, surgery, radiotherapy and naturopathic interventions as well as test treatments for treating non-small cell lung cancer. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

Moreover, a “treatment” or “prevention” regime of a subject with a therapeutically effective amount of the compound of the present disclosure may consist of a single administration, or alternatively comprise a series of applications.

The term “xenograft” as used herein, refers to cells, tissues, or organs that are the result of a transplantation of cells, tissues or organs from one species to another.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

### II. Methods and Apparatus

### A. Diagnostic Methods

It is demonstrated herein that different subtypes of non-small cell lung carcinoma have distinct biomarker signatures. For example, a number biomarkers show increased levels in non-small cell lung carcinoma of the adenocarcinoma (ADC) subtype and a number of biomarkers show increased expression in non-small cell lung carcinoma of the squamous cell carcinoma (SCC) subtype.

According to one aspect, the disclosure includes a method of screening for, diagnosing or detecting non-small cell lung carcinoma or an increased likelihood of developing non-small cell lung carcinoma in a subject comprising:
(a) determining a level of at least one biomarker associated with non-small cell lung carcinoma in a test sample from the subject, the at least one biomarker selected from the biomarkers set out in Table 2, 4A, 4B, 6 and/or 7; and
(b) comparing the level of the at least one biomarker in the test sample with a control;
   wherein detecting an difference in the level of the at least one biomarker in the test sample compared to the control is indicative of whether the subject has or does not have non-small cell lung carcinoma or an increased likelihood of developing non-small cell lung carcinoma.

In an embodiment, the biomarker is selected from the biomarkers set out in Table 2, Table 4A and Table 4B.

In an embodiment, the level of the biomarker determined is a polypeptide level or a nucleic acid level.

In an embodiment, the difference in the level of the biomarker is an increase in the level of the at least one biomarker in the test sample compared to a control. The control may be a sample from, or a numerical value that corresponds to, a control subject that does not have non-small cell lung carcinoma. In an embodiment, for biomarkers which are increased in non-small cell lung cancer, detecting an increased level is indicative the subject has non-small cell lung carcinoma or an increased risk of developing non-small cell lung carcinoma.

In yet a further embodiment, the level of at least 2, at least 3, at least 4, at least 5, at least 10, at least 14, at least 20, or at least 25 biomarkers is determined.

In an embodiment, the ratio of the level of the biomarker in the test sample compared to the control is greater than 2, 3, 5, 10, 12, 15, 20, 25, 30, 35, 40, 45, 50 or more. In an embodiment, an increased level of at least 2, at least 3, at least 4, at least 5, at least 10, at least 14, at least 20, or at least 25 biomarkers compared to the control is detected and/or is indicative of non-small cell lung carcinoma or an increased likelihood of developing non-small cell lung carcinoma in the subject.

In another embodiment, the level of at least one biomarker in the test sample is compared to a positive control in addition to or instead of a control. The positive control may be a sample from, or a numerical value that corresponds to, a subject or population of subjects known to have non-small cell lung carcinoma.

In an embodiment, a similar level or an increased level compared to the positive control is indicative of non-small cell lung carcinoma or an increased likelihood of developing non-small cell lung carcinoma in the subject.

In an embodiment, a decrease in the level of the biomarker in the test sample compared to the positive control is indicative the subject does not have non-small cell lung carcinoma or an increased risk of developing non-small cell lung carcinoma.

In an embodiment, the non-small cell lung carcinoma is adenocarcinoma or squamous cell carcinoma.

In another embodiment, an expression profile of the test sample obtained from the subject is determined. The expression profile comprises a level for each of at least two biomarkers associated with non-small cell lung carcinoma, these biomarkers being selected from the biomarkers set out in Table 2, 4A, 4B, 6 and/or 7. In this embodiment, the control is a reference profile associated with a non-small cell lung carcinoma subtype selected from adenocarcinoma and squamous cell carcinoma, and an expression profile most similar to the reference profile associated with adenocarcinoma is indicative that the subject has adenocarcinoma and an expression profile most similar to the reference profile associated with squamous cell carcinoma is indicative that the subject has squamous cell carcinoma.

The biomarker may be a keratin. In one embodiment, the keratin is selected from type KRT8, KRT18, KRT20, KRT7, KRT19, KRT5, KRT14, KRT15, KRT6A, KRT6B, KRT6C, KRT16, KRT17, KRT4, KRT13, KRT1, KRT10, KRT2, KRT3, KRT76, KRT78 and/or KRT80. In one embodiment, an increased level of KRT5, KRT6 and/or KRT15 is indicative that the subject has non-small cell lung cancer of the squamous cell carcinoma subtype. In one embodiment, an increased level of KRT7 is indicative that the subject has non-small cell lung cancer of the adenocarcinoma subtype.

In one embodiment, the biomarker is Carbamoyl-Phosphate Synthase (CPS-1) and an increased level is indicative that the subject has non-small cell lung cancer of the adenocarcinoma subtype.

In another embodiment, the biomarker is Anterior Gradient homolog 2 (AGR2) and an increased level is indicative that the subject has non-small cell lung cancer of the adenocarcinoma subtype.

In one embodiment, the biomarker is plakophilin-1 (PLP1) and an increased level is indicative that the subject has non-small cell lung cancer of the squamous cell carcinoma subtype.

In one embodiment, the expression profile comprises the expression level of at least two keratins.

According to another aspect, the disclosure also includes a method of differentiating between non-small cell lung carcinoma of the adenocarcinoma subtype and non-small cell lung carcinoma of the squamous cell carcinoma subtype in a subject, or detecting an increased likelihood of developing non-small cell lung carcinoma of the adenocarcinoma subtype or non-small cell lung carcinoma of the squamous cell carcinoma subtype in a subject comprising:
(a) determining a level of at least one biomarker associated with non-small cell lung carcinoma of the adenocarcinoma subtype or non-small cell lung carcinoma of the squamous cell subtype in a test sample from the subject wherein the at least one biomarker is selected from the biomarkers set out in Table 2, 4A, 4B, 6 and/or 7; and
(b) comparing the level of the at least one biomarker in the test sample with a control;
wherein detecting a difference or a similarity in the level of the at least one biomarker in the test sample compared to the control is indicative of whether the subject has non-small cell lung carcinoma of adenocarcinoma subtype or non-small cell lung carcinoma of squamous cell carcinoma subtype, or an increased likelihood of developing non-small cell lung carcinoma of adenocarcinoma subtype or non-small cell lung carcinoma of squamous cell carcinoma subtype.

Determination of non-small cell lung cancer subtype can involve classifying a subject with, or suspected of having non-small cell lung cancer based on the similarity of a subject's expression profile to one or more reference profiles associated with a particular lung cancer subtype. For example, the subject's expression profile can be compared to a reference profile associated with non-small cell lung cancer of the adenocarcinoma subtype and/or a reference profile associated with non-small cell lung cancer of the of the squamous cell carcinoma cell type.

Accordingly, in another aspect the disclosure includes a method of classifying a subject having or suspected of having non-small cell lung carcinoma as having adenomacarcinoma subtype or squamous cell carcinoma subtype, comprising:
a) obtaining a subject an expression profile of a sample from the subject;
b) obtaining a reference profile associated with a non-small cell lung carcinoma subtype, wherein the subject expression profile and the reference profile each have at least 2 values, each value representing the level of a biomarker, each biomarker selected from the biomarkers set out in Tables 2, 4A, 4B, 6 and/or 7; and
c) selecting the reference profile most similar to the subject expression profile, to thereby identify the non-small cell lung carcinoma subtype for the subject.

Wherein a plurality of biomarkers are assessed, the method can comprise calculating a measure of similarity. Accordingly, in an embodiment, the disclosure provides a method for classifying a subject having or suspected of having non-small cell lung cancer as having adenomacarcinoma subtype or squamous cell carcinoma subtype, comprising:
a) calculating a first measure of similarity between a first expression profile and an adenomacarcinoma subtype reference profile and a second measure of similarity between the first expression profile and a squamous cell carcinoma subtype reference profile; the first expression profile comprising the expression levels of a first plurality of genes in a sample from the subject; the adenomacarcinoma subtype reference profile comprising, for each gene in the first plurality of genes, the average expression level of the gene in a plurality of adenomacarcinoma subtype subjects; and the squamous cell carcinoma subtype reference profile comprising, for each gene in the first plurality of genes, the average expression level of the gene in a plurality of squamous cell carcinoma subtype subjects, the first plurality of genes comprising at least 2 of the genes listed in Table 2, 4A, 4B, 6 and/or 7; and
b) classifying the subject as having adenocarcinoma subtype if the first expression profile has a higher similarity to the good prognosis reference profile than to the squamous cell carcinoma subtype reference profile, or classifying the subject as squamous cell carcinoma subtype if the first expression profile has a higher similarity to the squamous cell carcinoma subtype reference profile than to the adenocarcinoma reference profile.

A number of algorithms can be used to assess similarity of samples. For example, similarity can be assessed by determining the Euclidean distance of a sample expression profile to a class centroid.

Wards algorithm can be used for forming hierarchical groups of mutually exclusive subsets of samples.

In an embodiment, the biomarker associated with non-small cell lung carcinoma of the adenocarcinoma subtype or non-small cell lung carcinoma of the squamous cell subtype is selected from the biomarkers set out in Table 2, Table 4A and Table 4B.

In an embodiment, the level of the at least one biomarker determined is a polypeptide level or a nucleic acid level.

In an embodiment, the altered level is an increase in the level of the biomarker in the test sample compared to a control. This control may be a sample from, or a numerical value that corresponds to, a control subject that does not have non-small cell lung carcinoma. The increase of the level of the biomarker is indicative of whether the subject has non-small cell lung carcinoma of the adenocarcinoma subtype or non-small cell lung carcinoma of the squamous cell carcinoma subtype, or an increased likelihood of developing non-small cell lung carcinoma of the adenocarcinoma subtype or non-small cell lung carcinoma of the squamous cell carcinoma subtype.

In yet a further embodiment, the level of at least 2 at least 3, at least 4, at least 5, at least 10, at least 15, at least 20, or at least 25 biomarkers is determined. In an embodiment, the ratio of the level of the biomarker in the test sample compared to the control is greater than 2, 3, 5, 10, 12, 15, 20, 25, 30, 35, 40, 45, 50 or more. In another embodiment, an increased level of at least 2, at least 3, at least 4, at least 5, at least 10, at least 15, at least 20, or at least 25 biomarkers compared to the control is detected and/or is indicative of non-small cell lung carcinoma of the adenocarcinoma subtype or non-small cell lung carcinoma of the squamous cell carcinoma subtype, or an increased likelihood of developing non-small cell lung carcinoma of the adenocarcinoma subtype or non-small cell lung carcinoma of the squamous cell carcinoma subtype.

In another embodiment, the level of the at least one biomarker in the test sample is compared to a positive control in addition to or instead of a control. This positive control may be a sample from, or a numerical value that corresponds to, a control subject with non-small cell lung carcinoma of the adenocarcinoma subtype or a control subject with non-small cell lung carcinoma of the squamous cell carcinoma subtype. A similar level or increased level compared to the positive control is indicative of non-small cell lung carcinoma of the adenocarcinoma subtype or non-small cell lung carcinoma of the squamous cell carcinoma subtype, or an increased likelihood of developing non-small cell lung carcinoma of the adenocarcinoma subtype or non-small cell lung carcinoma of the squamous cell carcinoma subtype. Alternatively, a decrease in the level of the biomarker in the test sample compared to the positive control is indicative the subject does not have non-small cell lung carcinoma of the adenocarcinoma subtype or non-small cell lung carcinoma of the squamous cell carcinoma subtype, or an increased likelihood of developing non-small cell lung carcinoma of the adenocarcinoma subtype or non-small cell lung carcinoma of the squamous cell carcinoma subtype.

In another embodiment, an expression profile in the test sample from the subject is determined. The expression profile comprises a level for each of at least two biomarkers associated with non-small cell lung carcinoma of the adenocarcinoma subtype or of the squamous cell carcinoma subtype, wherein the at least two biomarkers are selected from the biomarkers set out in Table 2, 4A, 4B, 6 and/or 7. In this embodiment, the control is a reference profile associated with a non-small cell lung carcinoma subtype selected from adenocarcinoma and squamous cell carcinoma, and an expression profile most similar to the reference profile associated with adenocarcinoma is indicative that the subject has adenocarcinoma and an expression profile most similar to the reference profile associated with squamous cell carcinoma is indicative that the subject has squamous cell carcinoma.

In one embodiment, an increased level of KRT5, KRT6 or KRT15 is indicative that the subject has non-small cell lung cancer of the squamous cell carcinoma subtype.

In one embodiment, an increased level of KRT7 is indicative that the subject has non-small cell lung cancer of the adenocarcinoma subtype.

In one embodiment, the biomarker is CPS-1 and an increased level is indicative that the subject has non-small cell lung cancer of the adenocarcinoma subtype.

In one embodiment, the biomarker is plakophilin-1 and an increased level is indicative that the subject has non-small cell lung cancer of the squamous cell carcinoma subtype.

In one embodiment, the expression profile comprises the expression level of at least two keratins.

According to another aspect, the disclosure also includes a method of screening for, diagnosing or detecting non-small cell lung carcinoma of the adenocarcinoma subtype or an increased likelihood of developing non-small cell lung carcinoma of the adenocarcinoma subtype in a subject comprising:

(a) determining a level of at least one biomarker associated with non-small cell lung carcinoma of the adenocarcinoma subtype in a test sample from the subject wherein the at least one biomarker is selected from the biomarkers set out in Table 2 or Table 4A; and

(b) comparing the level of the at least one biomarker in the test sample with a control;

wherein detecting an altered level of the at least one biomarker in the test sample compared to the control is indicative of whether the subject has or does not have non-small cell lung carcinoma of the adenocarcinoma subtype or an increased likelihood of developing non-small cell lung carcinoma of the adenocarcinoma subtype.

In an embodiment, the biomarker associated with non-small cell lung carcinoma of the adenocarcinoma subtype may be selected from the biomarkers set out in Table 2.

In an embodiment, the level of the at least one biomarker determined is a polypeptide level or a nucleic acid level.

In an embodiment, the altered level of the biomarker is an increase in the level of the at least one biomarker in the test sample compared to a control. This control may be a sample from, or a numerical value that corresponds to, a control subject that does not have non-small cell lung carcinoma or a control subject that does not have non-small cell lung carcinoma of the adenocarcinoma subtype. The increased level is indicative the subject has non-small cell lung carcinoma of the adenocarcinoma subtype or an increased risk of developing non-small cell lung carcinoma of the adenocarcinoma subtype.

In an embodiment, the level of at least 2, at least 3, at least 4, at least 5, at least 10, at least 15, at least 20, or at least 25 biomarkers is determined. In an embodiment, a ratio of the level of the biomarker in the test sample compared to the control is greater than 2, 3, 5, 10, 12, 15, 20, 25, 30, 35, 40, 45, 50 or more. In another embodiment, an increased level of at least 2, at least 3, at least 4, at least 5, at least 10, at least 15, at least 20, or at least 25 biomarkers compared to the control is detected and/or is indicative of non-small cell lung carcinoma of the adenocarcinoma subtype or an increased likelihood of developing non-small cell lung carcinoma of the adenocarcinoma subtype in the subject.

In another embodiment, the level of the biomarker in the test sample is compared to a positive control in addition to or instead of a control. This positive control may be a sample from, or a numerical value that corresponds to, a control subject with non-small cell lung carcinoma of the adenocarcinoma subtype. A similar or increased level compared to the positive control is indicative of non-small cell lung carcinoma of the adenocarcinoma subtype or an increased likelihood of developing non-small cell lung carcinoma of the adenocarcinoma subtype in the subject. Alternatively, a decrease in the level of the biomarker in the test sample compared to the positive control, wherein the decreased level is indicative the subject does not have non-small cell lung carcinoma of the adenocarcinoma subtype or an increased risk of developing non-small cell lung carcinoma of the adenocarcinoma subtype.

In an embodiment, an expression profile in the test sample from the subject is determined. The expression profile comprises a level for each of at least two biomarkers associated with non-small cell lung carcinoma of the adenocarcinoma subtype, wherein the at least two biomarkers are selected from the biomarkers set out in Table 2 or Table 4A. In this embodiment, the control is a reference profile associated with a non-small cell lung carcinoma of the adenocarcinoma subtype, and an expression profile most similar to the reference profile associated with non-small cell lung carcinoma of the adenocarcinoma subtype is indicative that the subject has non-small cell lung carcinoma of the adenocarcinoma subtype.

In an embodiment, the at least one biomarker is a keratin. The keratin may be selected from KRT18, KRT7, KRT14 or KRT17. In an embodiment, the keratin is KRT7.

In a further embodiment, an increased level of KRT7 is indicative that the subject has non-small cell lung cancer of the adenocarcinoma subtype.

In a further embodiment, the biomarker is CPS-1 and an increased level is indicative that the subject has non-small cell lung cancer of the adenocarcinoma subtype.

In an embodiment, the expression profile comprises the expression level of at least two keratins.

According to another aspect, the disclosure also includes a method of screening for, diagnosing or detecting non-small cell lung carcinoma of the squamous cell carcinoma subtype or an increased likelihood of developing non-small cell lung carcinoma of the squamous cell carcinoma subtype in a subject comprising:
(a) determining a level of at least one biomarker associated with non-small cell lung carcinoma of the squamous cell carcinoma subtype in a test sample from the subject wherein the at least one biomarker is selected from the biomarkers set out in Table 2 or Table 4B; and
(b) comparing the level of the at least one biomarker in the test sample with a control;

wherein detecting a difference or similarity in the level of the at least one biomarker in the test sample compared to the control is indicative of whether the subject has or does not have non-small cell lung carcinoma of the squamous cell carcinoma subtype or an increased likelihood of developing non-small cell lung carcinoma of the squamous cell carcinoma subtype. In an embodiment, the biomarker associated with non-small cell lung carcinoma of the squamous cell carcinoma subtype is selected from the biomarkers set out in Table 2.

In an embodiment, the level of the biomarker determined is a polypeptide level or a nucleic acid level.

In an embodiment, the difference in the level is an increase in the level of the biomarker in the test sample compared to a control. This control may be a sample from, or a numerical value that corresponds to, a control subject that does not have non-small cell lung carcinoma or a control subject that does not have non-small cell lung carcinoma of the squamous cell carcinoma subtype. An increased level is indicative the subject has non-small cell lung carcinoma of the squamous cell carcinoma subtype or an increased risk of developing non-small cell lung carcinoma of the squamous cell carcinoma subtype.

In an embodiment, the level of at least 2, at least 3, at least 4, at least 5, at least 10, at least 15, at least 20, or at least 25 biomarkers is determined.

In an embodiment, a ratio of the level of the biomarker in the test sample compared to the control is greater than 2, 3, 5, 10, 12, 15, 20, 25, 30, 35, 40, 45, 50 or more.

In an embodiment, an increased level of at least 2, at least 3, at least 4, at least 5, at least 10, at least 15, at least 20, or at least 25 biomarkers compared to the control is detected and/or indicative of non-small cell lung carcinoma of the squamous cell carcinoma subtype or an increased likelihood of developing non-small cell lung carcinoma of the squamous cell carcinoma subtype in the subject.

In another embodiment, the level of the biomarker in the test sample is compared to a positive control in addition to or instead of a control. This positive control may be a sample from, or a numerical value that corresponds to, a control subject with non-small cell lung carcinoma of the squamous cell carcinoma subtype. A similar or increased level compared to the positive control is indicative of non-small cell lung carcinoma of the squamous cell carcinoma subtype or an increased likelihood of developing non-small cell lung carcinoma of the squamous cell carcinoma subtype in the subject. Alternatively, a decrease in the level of the biomarker in the test sample compared to the positive control is indicative the subject does not have non-small cell lung carcinoma of the squamous cell carcinoma subtype or does not have an increased risk of developing non-small cell lung carcinoma of the squamous cell carcinoma subtype.

In an embodiment, an expression profile of the test sample from the subject is determined. The expression profile comprises a level for each of at least two biomarkers associated with non-small cell lung carcinoma squamous cell carcinoma, the at least two biomarkers selected from the biomarkers set out in Table 2 or Table 4B. In this embodiment, the control is a reference profile associated with a non-small cell lung carcinoma of the squamous cell carcinoma subtype, and an expression profile most similar to the reference profile associated with non-small cell lung carcinoma of the squamous cell carcinoma subtype is indicative that the subject has non-small cell lung carcinoma of the squamous cell carcinoma subtype.

In an embodiment, the at least one biomarker is a keratin.

In an embodiment, the biomarker is plakophilin-1 and an increased level is indicative that the subject has non-small cell lung cancer of the squamous cell carcinoma subtype.

In an embodiment, the level of at least one biomarker or the expression product is determined using mass spectrometry.

In an embodiment, the mass spectrometry comprises tandem mass spectrometry, 1D LC MS/MS, 2D LC MS/MS, SRM or MRM.

In an embodiment, the biomarker level is detected by summing the spectral counts for biomarker peptides. Spectral counts for peptides corresponding to a single protein can be summed and protein level counts can be normalized by obtaining the relative abundance ratio (dividing by the total sample spectral counts) than multiplying by the overall experimental spectral count.

In an embodiment, the biomarker is a biomarker listed in Table 6 and mass spectrometry is used to detect a peptide listed in Table 6.

The methods described herein can be computer implemented. In an embodiment, the method further comprises: (c) displaying or outputting to a user interface device, a computer readable storage medium, or a local or remote computer system; the classification produced by the classifying step (b). In another embodiment, the method comprises displaying or outputting a result of one of the steps to a user interface device, a computer readable storage medium, a monitor, or a computer that is part of a network.

### B. Method of Treatment

According to another aspect, the disclosure also includes a method of treating non-small cell lung carcinoma in a subject comprising:
a) diagnosing non-small cell lung carcinoma in a test sample from the subject; and
b) administering a treatment suitable for the treatment of non-small cell lung carcinoma to the subject.

According to another aspect, the disclosure also includes a method of treating non-small cell lung carcinoma of the subtype adenocarcinoma or non-small cell lung carcinoma of the subtype squamous cell carcinoma in a subject comprising:
a) diagnosing non-small cell lung carcinoma ofadenocarcinoma subtype or non-small cell lung carcinoma of squamous cell carcinoma subtype in a test sample from the subject; and
b) administering to the subject a treatment suitable for treating non-small cell lung carcinoma of adenocarcinoma subtype when adenocarcinoma subtype is detected or administering a treatment suitable for treating non-small cell lung carcinoma of squamous cell carcinoma subtype when squamous cell carcinoma subtype is detected.

According to another aspect, the disclosure also includes a method of treating non-small cell lung carcinoma of adenocarcinoma subtype in a subject comprising:
a) diagnosing non-small cell lung carcinoma of adenocarcinoma subtype in the subject; and
b) administering a treatment suitable for treating non-small cell lung carcinoma of adenocarcinoma subtype to the subject.

A method of treating non-small cell lung carcinoma of squamous cell carcinoma subtype in a subject comprising:
a) diagnosing a non-small cell lung carcinoma of squamous cell carcinoma subtype in a test sample from the subject; and
b) administering a treatment suitable for treating non-small cell lung carcinoma of squamous cell carcinoma subtype to the subject.

### C. Method for Quantifying a Biomarker

According to another aspect, the disclosure also includes a SRM/MRM method for quantifying a level of at least one biomarker associated with non-small cell lung carcinoma in a sample, the method comprising the steps of:
a) isotope labeling a peptide fragment of the at least one biomarker wherein the at least one biomarker is selected from the biomarkers set out in Table 2, 4A, 4B, 6 and/or 7; and

b) evaluating the biomarker level using SRM/MRM mass spectrometry. In an embodiment, the amount of biomarker peptide fragment present in a sample can be determined by summing the area from all transitions and normalizing the totals to an area obtained for a control peptide for example, the peptide LISWYDNEFGYSNR (SEQ ID NO:1) that is found in GAPDH.

In an embodiment, the level of a biomarker listed in Table 6 is quantified using SRM/MRM mass spectrometry. In an embodiment, the peptide fragment that is isotope labeled, is a peptide fragment listed in Table 6. In an embodiment, the level of epidermal growth factor receptor, optionally phosphorylated epidermal growth factor receptor is quantified using SRM/MRM mass spectrometry.

In an embodiment, the EGFR peptide fragment detected is NLQEILHGAVR (SEQ ID NO:12).

In an embodiment, the method comprises a dynamic detection range corresponding from about 10 000 copies to about 1 million copies of per cell.

The quantifying method may also further comprise the step of determining activation of the epidermal growth factor receptor (EGFR) network.

It is demonstrated herein that SRM/MRM is able to more accurately assess the level of for example EGFR and the accurate quantification of EGFR protein levels by SRM may enable the further stratification of NSCLC in terms of EGFR levels, beyond what has been achieved by IHC, measures of gene copy number, and mutations.

According to another aspect, the disclosure also includes a method for treating non-small cell lung carcinoma in a subject comprising:

a) quantifying a level of EGFR in a test sample from the subject; and

b) administering an EGFR directed drug to the subject when the level of EGFR level quantified is above a threshold indicative that the subject would benefit from the EGFR directed drug.

In an embodiment, one or more parameters provided in Example 1 are used.

### D. Kits

According to another aspect, the disclosure also includes a kit for measuring the level of at least one biomarker associated with non-small cell lung cancer or a subtype thereof in a sample, wherein the at least one biomarker is selected from the biomarkers set out in Table 2, 4A, 4B, 6 and/or 7, comprising:
a) a biomarker specific reagent, labeling isotope and/or a peptidase such as trypsin;
b) a kit control, optionally a peptide fragment of a biomarker;
c) an array slide; and
d) optionally instructions for use.

In an embodiment, the at least one biomarker is selected from the biomarkers set out in Table 2, Table 4A or 4B.

In an embodiment, the at least one biomarker is a keratin. In an embodiment, the kit comprises a set of biomarker detection agents for detecting a set of keratin biomarkers. In embodiment, the array slide comprises a set of biomarker detection agents for detecting a set of keratin biomarkers.

In an embodiment, the keratin is selected from KRT8, KRT18, KRT20, KRT7, KRT19, KRT5, KRT14, KRT15, KRT6A, KRT6B, KRT6C, KRT16, KRT17, KRT4, KRT13, KRT1, KRT10, KRT2, KRT3, KRT76, KRT78 and/or KRT80.

In an embodiment, the keratin is selected from KRT18, KRT7, KRT5, KRT14, KRT15, KRT6A, KRT16, KRT17, KRT4 and/or KRT13.

In an embodiment, the at least one biomarker is CPS-1.

In an embodiment, the at least one biomaker is AGR2.

In an embodiment, the at least one biomarker is plakophilin-1.

In an embodiment, the biomarker specific reagent is an antibody or antibody fragment.

In an embodiment, the biomarker specific reagent is a probe, or primer set that amplifies a nucleic acid transcript of the biomarker.

In an embodiment, the control is a peptide fragment of the at least one biomarker.

The kit can comprise for example, specimen collection tubes for example for collecting a biopsy, extraction buffer, positive controls, and the like.

The following non-limiting examples are illustrative of the present disclosure:

### III. Examples

### Xenograft Tumor Generation and Pathology

Routinely harvested fresh human NSCLC were resected surgically at The University Health Network (Toronto) and directly implanted into non-obese diabetic and severe combined immune-deficient (NOD-SCID) mice to establish primary tumor xenograft models. Each tumor model was verified by at least three serial in vivo passages to demonstrate engraftment stability. OncoCarta MassARRAY Chip (Sequenom) mutation screening was conducted to detect mutations in the EGFR, KRAS, and PIK3CA genes. These results were validated by direct DNA sequencing of tumor xenograft specimens.

### Immunohistochemistry

Formalin-fixed paraffin-embedded (FFPE) tissue blocks were cut at 4 um thickness onto slides and dried in 60° C. oven overnight. Slides were further processed and stained in a fully automated process using the BenchMark XT (Ventana Medical Systems Inc.). Slides were scored by a pathologist (NY). Staining intensity was scored positive or negative. When specimens showed partial positive labeling, the percentage of tumor cells labeled is estimated.

### Sample Preparation and Western Immuno Blotting

Xenograft tissues were harvested from mice and immediately stored in liquid nitrogen. Aliquots of tissue (approximately 50 mg) were mixed with lysis buffer (1 ml buffer per 10 mg tissue; 20 mM HEPES pH 8.0, 9 M Urea, 1 mM sodium orthovanadate, 2.5 mM sodium pyrophosphate, 1 mM β-glycerophosphate) and subjected to ultra-sonication for 1 min, followed by centrifugation (20,000×g) for 20 min. Aliquots of supernatants (clarified lysates; 50 μl) were set aside for Western blotting analysis. Routinely, the concentrations of clarified lysates were approximately 2 mg/ml (protein). An equal volume of 2×SDS-PAGE sample loading buffer was added, and the samples were resolved by standard SDS-PAGE methods, and then electrophoretically transferred to Immobilon-P membranes (Millipore) for Western blotting, essentially as described previously ¹⁰.

For MS analysis, clarified lysates were reduced with 4.5 mM DTT, carboxamidomethylated by using 10 mM iodoacetamide, diluted 4-fold, digested by incubation with Trypsin-TPCK for 12 h. Peptides were then desalted by using C18 resin as described previously ¹¹. The eluted peptides were aliquoted and lyophilized. 2 μg or 15 μg dried, desalted peptides were dissolved in 0.1% formic acid and analyzed by 1D or 2D LC/MS/MS, respectively.

### 1D LC-MS/MS

All samples were analyzed on a LTQ-Orbitrap XL. The instrument method consisted of one MS full scan (400-1800 m/z) in the Orbitrap mass analyzer, an AGC target of 500,000 with a maximum ion injection of 500 ms, 1 microscan and a resolution of 60,000 and using the preview scan option. Six data-dependent MS/MS scans were performed in the linear ion trap using the three most intense ions at 35% normalized collision energy. The MS and MS/MS scans were obtained in parallel. AGC targets were 10,000 with a maximum ion injection time of 100 ms. A minimum ion intensity of 1,000 was required to trigger a MS/MS spectrum. The dynamic exclusion was applied using a maximum exclusion list of 500 with one repeat count with a repeat duration of 30 seconds and exclusion duration of 45 seconds.

2D LC-MS/MS analyses

A fully automated 4-step two-dimensional chromatography sequence was set up as previously described ¹². Peptides were loaded on a 7 cm pre-column (150 μm i.d.) containing a Kasil frit packed with 3.5 cm 5μ Magic C18 100 Å reversed phase material (Michrom Bioresources) followed by 3.5 cm Luna® 5μ SCX 100 Å strong cation exchange resin (Phenomenex, Torrance, Calif.). Samples were automatically loaded from a 96-well microplate autosampler using an EASY-nLC system (Proxeon Biosystems, Odense, Denmark) at 3 μl/minute. The pre-column was connected to an 8 cm fused silica analytical column (75 μm i.d.) via a micro splitter tee (Proxeon) to which a distal 2.3 kV spray voltage was applied. The analytical column was pulled to a fine electrospray emitter using a laser puller. For the peptide separation on the analytical column a water/acetonitrile gradient was applied at an effective flow rate of 400 nl/minute, controlled by the EASY-nLC. Ammonium acetate salt bumps (8 μl) were applied at the following concentrations (0 mM, 100 mM, 300 mM and 500 mM), using the 96-well micro plate autosampler at a flow-rate of 3 ml/minute in a vented-column set-up.

### SRM

SRM was carried out on duplicate 5 μg aliquots of each xenograft lysate. The peptides were captured on a 150 μm ID C18 pre-column and separated over a 75 μm ID analytical column constructed with an emitter tip. The separation was carried out with a gradient of 0 to 65% acetonitrile in 0.1% formic acid over 40 min using the EASY-nLC split-free HPLC system. The eluted peptides were monitored by using a TSQ Quantum Vantage triple quadrupole mass spectrometer (ThermoFisher, San Jose, Calif.). The dwell time was 20 ms and the scan width was 0.01 amu. The S-lens was varied with precursor m/z values and a 10 V declustering potential was used. Q1 and Q3 resolution were set to 0.2 and 0.7 amu, respectively. The transitions used are shown in Table 6. To normalize for the amount of human peptides present in each sample, the area from all transitions were summed and the totals were normalized to the areas obtained for the peptide LISWYDNEFGYSNR (SEQ ID NO:1) that is found only in human GAPDH. Note that the 2D LC-MS/MS experiments verified that human GAPDH was not statistically different in abundance between ADC and SCC samples.

Therefore, the SRM value for human GAPDH peptide LISWYDNEFGYSNR (SEQ ID NO:1), based on 3 transitions, which averaged 5.7±0.8 (SE)×10⁵ units across the ten samples, was used to normalize the summed SRM transition measurements associated with individual peptides from corresponding xenograft samples. Collision energy was calculated by using the formula 3.41+0.034×(m/z of parent peptide), with collision gas pressure at 1.5 mTorr as described by Prakash and colleagues. ³⁴

### Clustering and Identification of Differentially Expressed Proteins

Spectral counts for peptides corresponding to a single protein were summed and protein level counts were normalized by obtaining the relative abundance ratio (dividing by the total sample spectral counts) than multiplying by the overall experimental spectral count. Spectra count values of zero were changed to 0.2 as part of the normalization routine similar, as recently reported ¹³⁻¹⁶. For clustering analysis, the 3 replicates for each sample were averaged, and the normalized data was then filtered to include proteins which were detected in at least 2 samples in the entire data set of normalized protein data (i.e. sample incidence ≧2). Hierarchical clustering was applied to the normalized protein data in R (v2.10.0) via the ‘hclust’ function using Spearman's rank correlation as distance metrics and the ‘average’ agglomeration method, and was used to generate dendrograms. Heatmap plots were generated using the heatmap.2 function in the R package ‘gplots’ (v2.7.4), utilizing the log 2 transformed normalized protein data.

The Wilcoxon Rank Sum Test (‘wilcox.test’ in R (v2.10.0)) was used to identify differentially expressed proteins between ADC and SCC samples in the normalized protein data. Significance was assumed as p-value <0.05.

### Protein Identification and Data Analysis

Raw data was converted to m/z XML using ReAdW and searched by X!Tandem against a locally installed version of a merged human and mouse IPI (http://www.ebi.ac.uk/IPI) protein sequence database (version 3.54; 75,427 human sequences and 55,985 mouse sequences). The searches were performed with a fragment ion mass tolerance of 0.4 Da, a parent ion mass tolerance of ±10 ppm. Complete tryptic digest was assumed. Carbamidomethylation of cysteine was specified as fixed, and oxidation of methionine as variable modification.

To estimate and minimize the false positive rate the merged human and mouse protein sequence database also contained every IPI protein sequence in its reversed amino acid orientation (target-decoy strategy; total database size 262,824 sequences) as recently described ¹⁷′ ¹⁸. For the presented study, the value of total reverse spectra to total forward spectra was set to 0.5%, resulting in zero decoy sequences in the final output (0 reverse proteins for both the human and mouse assignments). Only fully tryptic peptides ≧7 amino acids, matching these criteria were accepted to generate the final list of identified proteins. Only proteins identified with two unique peptides per analyzed sample were accepted (i.e. 3 MudPITs per xenograft). To minimize protein inference, a database grouping scheme was developed, and only proteins with substantial peptide information were reported, as recently reported ^{14, 17, 19}. For a protein to be assigned “human or mouse” it required at least one unique peptide mapping uniquely to either a human or mouse entry in the mixed-species database.

### Results

### Experimental Plan

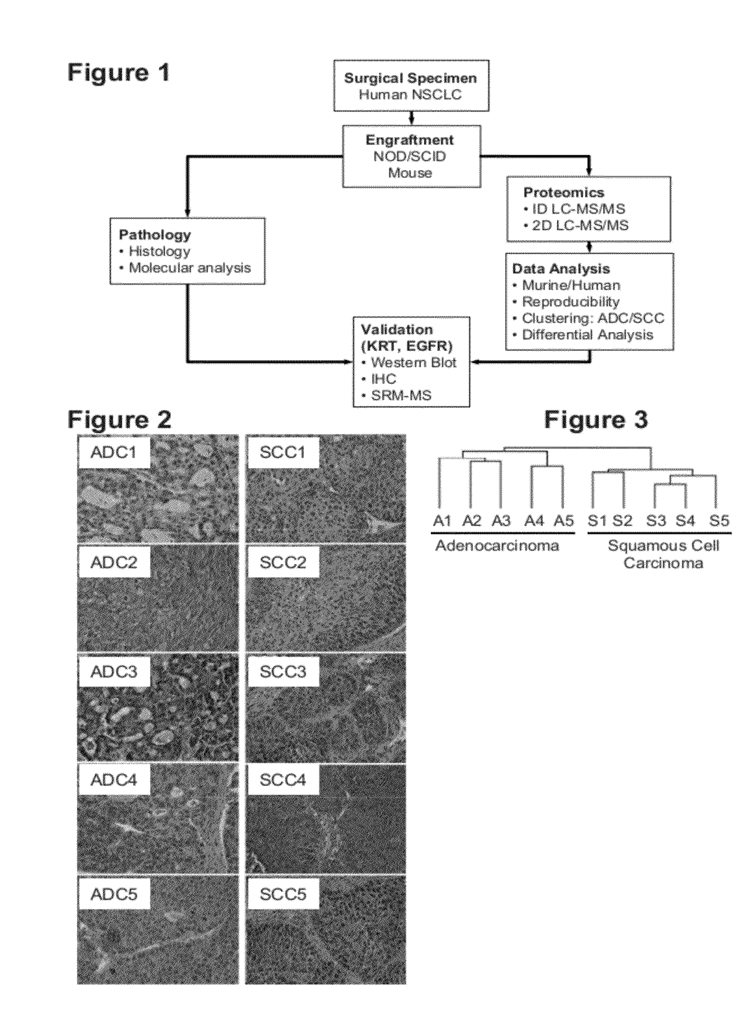

As part of a larger effort to establish and characterize >100 primary NSCLC xenografts, a pilot study was conducted with 10 NSCLC xenografts to test if a proteomics platform could be effectively applied to characterize these tumors (FIG. 1). The analytical proteomics platform included tandem MS analysis of tryptic peptides resolved by one dimensional (1D) or two dimensional (2D), nano scale liquid chromatography. This provided peptide and protein identifications and relative semi-quantification based on MS/MS spectral counting. In parallel, the xenografts were subjected to standard laboratory histopathology, which dictated their classification as ADC or SCC subtype. Analysis of MS data was completed with the objective to identify protein expression signatures characteristic of the ADC and SCC subtypes. Validation of protein expression involved limited applications of SRM-MS, IHC and Western immuno blotting.

### Xenograft Tumor Pathology

The establishment of the xenograft tumors and sample preparation are described above. Table 1 presents a summary of tumor information including limited histological and molecular features.

TABLE 1
NSCLC tumor xenograft histopathology and molecular features

| Xeno- | Subtype | | | Immunohistochemistry² | | | | | | |
|---|---|---|---|---|---|---|---|---|---|---|
| graft | and Cellular | | | | EGFR | | | | | |
| ID | Differentiation | Cellularity¹ | Mutations | EGFR | pY1068 | KRT5/6 | KRT7 | KRT14 | KRT19 | HMWK |
| ADC1 | Adeno, | 70-75 | — | 100% | 20% | — | 100% | — | 100% | — |
| | moderate | | | | | | | | | |
| ADC2 | Adeno, | >90 | — | 100% | 100% | — | 100% | 100% | 2-3% | — |
| | poor | | | | | | | | | |
| ADC3 | Adeno, | 70-75 | EGFR | 100% | 40% | — | 100% | — | 70% | — |
| | moderate | | Δ746-750 | | | | | | | |
| ADC4 | Adeno, | 70 | KRAS | 10% | 50% | — | 100% | — | 100% | — |
| | poor | | G12C | | | | | | | |
| ADC5 | Adeno, | >90 | KRAS | 50% | — | — | 100% | — | 40% | — |
| | poor | | G12D | | | | | | | |
| SCC1 | Squam, | >90 | — | 60% | 30% | 100% | — | 40% | 100% | 100% |
| | well | | | | | | | | | |
| SCC2 | Squam, | 80 | PIK3CA | 100% | — | 100% | — | 100% | 100% | 100% |
| | well | | E542K | | | | | | | |
| SCC3 | Squam, | 80-90 | — | 100% | 40% | 100% | 10% | — | 100% | 100% |
| | moderate | | | | | | | | | |
| SCC4 | Squam, | 85-90 | PIK3CA | 70% | — | 100% | — | 100% | 100% | 100% |
| | poor | | E545K | | | | | | | |
| SCC5 | Squam, | >90 | — | 100% | — | 100% | — | 70% | 100% | 100% |
| | moderate | | | | | | | | | |

¹Estimated tumor cell abundance among cells present
²Percentage positively stained cells

The ten xenografts were classified as ADC or SCC based on the histologies of the primary tumors and corresponding xenografts; there was good concordance in differentiation grades (FIG. 2). The tumors were screened for mutations by the OncoCarta™ v.1 MassARRAY system (Sequenom, San Diego, Calif.), and mutations were confirmed by sequencing. Five tumors were found to have activating, coding mutations in the EGFR, KRAS, and PIK3CA genes (Table 1).

### Analysis of NSCLC Xenograft Proteomes by 1D and 2D LC-MS/MS 1D LC-MS/MS

A relatively rapid (i.e. approximately 2 h per sample analysis), 1D LC-MS/MS approach was used to complete an initial analysis of proteins expressed in the 10 xenograft samples. This was followed by a more comprehensive, but time consuming (i.e. 8 h per sample analysis) 2D analysis (described below) ¹². Results were tabulated as normalized MS/MS spectral counts per assigned gene product (see Experimental Procedures) Variability in protein detection and expression were assessed by comparing results of technical and biological replicates at the protein and peptide levels. When a sample (ADC1) was analyzed in triplicate 493 proteins out of a total of 564, were identified in each replicate, indicating an overlap of 87%. To assess biological variation, equivalent portions of ADC1 were expanded in two different recipient mice, and then analyzed. The overlap between the two samples was 88% at the protein level: 491 proteins out of a total of 559, were found in both samples. These numbers suggested a reasonable degree of reproducibility at the protein level between samples analyzed by 1D LC-MS/MS.

The 1D LC-MS/MS analysis of the 10 samples was considered a preliminary scan of the NSCLC xenograft proteomes, and allowed identification of 635 proteins. See Experimental Procedures for protein identification criteria. As shown in the dendogram in FIG. 3, even by the relatively low resolution (in terms of proteome coverage) approach, hierarchical clustering of proteins based on normalized spectral counts separated the proteomes into two sets corresponding to the ADC and SCC subtypes (FIG. 3). In order to semi-quantify proteins and examine differential expression between the ADC and SCC subtypes, normalized spectral counts for proteins were summed across each subtype and compared. A subset of 57 proteins were significantly differentially expressed between the ADC and SCC subtypes. Ten proteins were deemed highly differentially expressed, and displayed a >10-fold increase or decrease between ADC and SCC (Table 2).

TABLE 2
Highly differentially expressed proteins in ADC and SCC
xenografts detected by 1D LC-MS/MS protein profiling

| Identified | ADC/SCC | SCC/ADC | |
|---|---|---|---|
| Proteins (635) | Ratio | Ratio | p-value |
| CPS1 | 24.3 | 0.04 | 0.049 |
| KRT7 | 13.6 | 0.07 | 0.001 |
| AGR2 | 10.1 | 0.10 | 0.017 |
| KRT14 | 0.08 | 11.9 | 0.022 |
| KRT17 | 0.07 | 14.7 | 0.0001 |
| KRT15 | 0.03 | 31.2 | 0.002 |
| KRT16 | 0.03 | 31.6 | 0.011 |
| KRT5 | 0.02 | 53.0 | 0.00003 |
| KRT6A | 0.02 | 59.1 | 0.00005 |
| KRT6B | 0.02 | 59.4 | 0.00006 |

Prominent among the highly differentially expressed proteins were 8 keratin (KRT) gene products, with KRT7 highly expressed in ADC, and seven others that were more highly expressed in SCC. The other two proteins found more highly expressed in ADC compared to SCC were the urea cycle component Carbamoyl-Phosphate Synthase (CSP1), and Anterior Gradient homolog 2 (AGR2). These data indicate that the 1D platform was sufficient to resolve ADC and SCC subtypes based on their significantly different proteomes.

### 2D LC-MS/MS Analysis

In order to increase statistical significance and proteome coverage, a more rigorous protocol involving triplicate analysis by 2D LC-MS/MS, MudPIT (Multidimensional Protein Identification Technology) was applied ^{12, 20, 21}. Each sample was analyzed in triplicate and MS/MS spectral counts tabulated essentially as described in Experimental Procedures ¹³⁻¹⁶. As a product of the 30 individual 2D analyses, 2015 proteins were identified (Table 3).

TABLE 3
NSCLC Proteomics Profiling Summary

| Xenograft | Identified Human | Xenograft | Identified Human |
|---|---|---|---|
| Name | Proteins | Name | Proteins |
| ADC1_1 | 628 | SCC1_1 | 701 |
| ADC1_2 | 650 | SCC1_2 | 696 |
| ADC1_3 | 649 | SCC1_3 | 708 |
| ADC1_Total | 671 | SCC1_Total | 738 |
| ADC2_1 | 890 | SCC2_1 | 611 |
| ADC2_2 | 830 | SCC2_2 | 620 |
| ADC2_3 | 811 | SCC2_3 | 612 |
| ADC2_Total | 933 | SCC2_Total | 645 |
| ADC3_1 | 1140 | SCC3_1 | 695 |
| ADC3_2 | 1185 | SCC3_2 | 691 |
| ADC3_3 | 1022 | SCC3_3 | 690 |
| ADC3_Total | 1271 | SCC3_Total | 719 |
| ADC4_1 | 634 | SCC4_1 | 825 |
| ADC4_2 | 630 | SCC4_2 | 797 |
| ADC4_3 | 545 | SCC4_3 | 769 |
| ADC4_Total | 663 | SCC4_Total | 854 |
| ADC5_1 | 698 | SCC5_1 | 665 |
| ADC5_2 | 686 | SCC5_2 | 645 |
| ADC5_3 | 685 | SCC5_3 | 665 |
| ADC5_Total | 734 | SCC5_Total | 692 |
| | | Total: | 2015 |

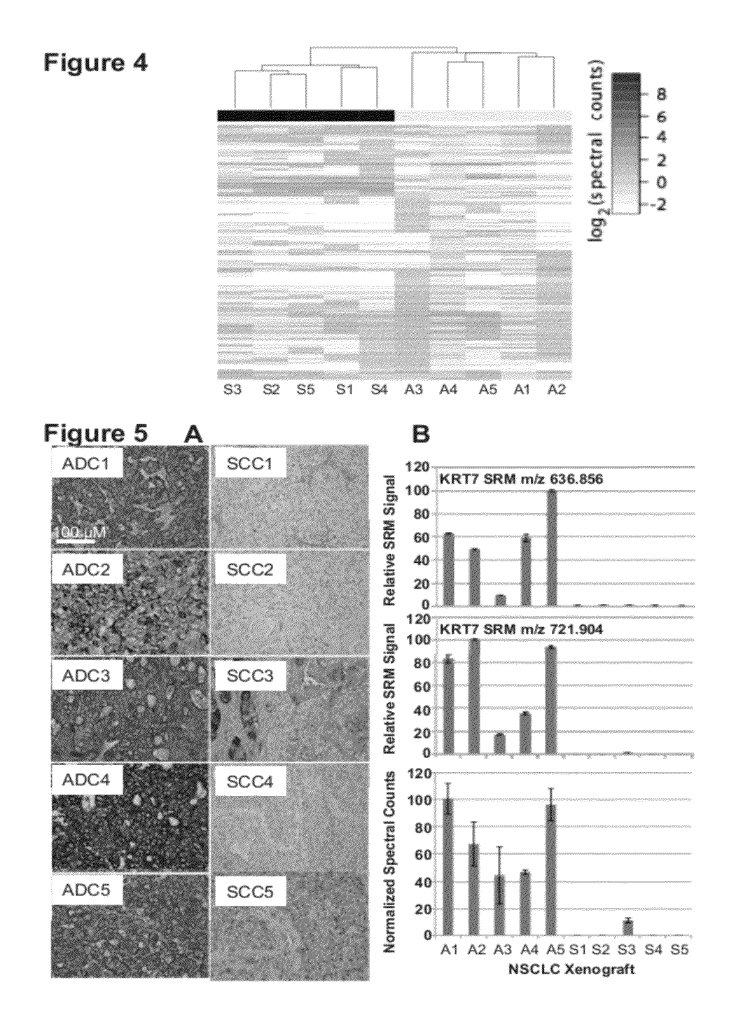

Expressed human proteins were subjected to hierarchical clustering analysis to determine if the ADC and SCC proteomes were distinct. FIG. 4 shows the results of hierarchical clustering of identified human proteins. Similar to the 1D results described above, the 2D dataset clustered into separate ADC and SCC sets.

By application of the Wilcoxon test, 178 human proteins were significantly different in their average expression between the ADC and SCC subtypes (Table 7). Within this set, 50 proteins were increased or decreased >10-fold in ADC compared with SCC xenografts (Table 4 A and 4B).

TABLE 7
Biomarkers differentially expressed in ADC and SCC

| | | IPI Accession |
|---|---|---|
| gene name | pvalue | Number |
| ACLY | 0.031746032 | IPI00021290.5 |
| ADH7 | 0.007936508 | IPI00028066.2 |
| AGR2 | 0.007936508 | IPI00007427.2 |
| AHNAK | 0.007936508 | IPI00021812.2 |
| AIFM1 | 0.015873016 | IPI00000690.1 |
| AK2 | 0.015873016 | IPI00215901.1 |
| ALDH18A1 | 0.015873016 | IPI00008982.1 |
| ALDH1B1 | 0.015873016 | IPI00103467.4 |
| ANXA3 | 0.007936508 | IPI00024095.3 |
| AP2A2 | 0.015873016 | IPI00016621.7 |
| ATIC | 0.007936508 | IPI00289499.3 |
| ATP1B3 | 0.007936508 | IPI00008167.1 |
| ATP5A1 | 0.031746032 | IPI00440493.2 |
| BCAP31 | 0.031746032 | IPI00218200.8 |
| BSG | 0.031746032 | IPI00019906.1 |
| C3 | 0.015873016 | IPI00783987.2 |
| CALML3 | 0.007936508 | IPI00216984.5 |
| CAND1 | 0.007936508 | IPI00100160.3 |
| CAPN1 | 0.031746032 | IPI00011285.1 |
| CCT8 | 0.031746032 | IPI00302925.4 |
| CD9 | 0.015873016 | IPI00215997.5 |
| CDH1 | 0.015873016 | IPI00000513.1, IPI00025861.3 |
| CES1 | 0.007936508 | IPI00010180.4, IPI00607693.2 |
| CKAP5 | 0.031746032 | IPI00028275.2 |
| CNDP2 | 0.031746032 | IPI00177728.3 |
| COASY | 0.015873016 | IPI00184821.1 |
| COPA | 0.015873016 | IPI00295857.7 |
| CPT2 | 0.015873016 | IPI00012912.1 |
| CRABP2 | 0.015873016 | IPI00216088.3 |
| CRIP2 | 0.015873016 | IPI00006034.1 |
| CRYZ | 0.031746032 | IPI00000792.1 |
| CTNND1 | 0.007936508 | IPI00182469.3 |
| CYP2S1 | 0.007936508 | IPI00164018.5 |
| DDAH2 | 0.015873016 | IPI00000760.1 |
| DDX24 | 0.015873016 | IPI00006987.1 |
| DHRS7 | 0.007936508 | IPI00006957.3 |
| DIAPH1 | 0.031746032 | IPI00030876.7 |
| DNAJC10 | 0.015873016 | IPI00293260.5 |
| DSC3 | 0.007936508 | IPI00031549.5 |
| DSG2 | 0.015873016 | IPI00028931.2 |
| DSG3 | 0.007936508 | IPI00031547.1 |
| DSP | 0.007936508 | IPI00013933.2 |
| DTYMK, LOC727761 | 0.007936508 | IPI00013862.7 |
| EIF3B | 0.007936508 | IPI00396370.6 |
| EPPK1 | 0.031746032 | IPI00010951.2 |
| ERLIN1 | 0.015873016 | IPI00007940.6 |
| FKBP10 | 0.015873016 | IPI00303300.3 |
| FLOT1 | 0.015873016 | IPI00027438.2 |
| GALE | 0.007936508 | IPI00553131.2 |
| GALK1 | 0.015873016 | IPI00019383.2 |
| GALNT6 | 0.015873016 | IPI00026991.4 |
| GALNT7 | 0.015873016 | IPI00328391.3 |
| GAR1 | 0.007936508 | IPI00302176.5 |
| GBP6 | 0.031746032 | IPI00375746.4 |
| GCN1L1 | 0.031746032 | IPI00001159.10 |
| GFPT1 | 0.007936508 | IPI00217952.6 |
| GLRX | 0.007936508 | IPI00219025.3 |
| GORASP2 | 0.015873016 | IPI00743931.3, IPI00916299.1 |
| GPC1 | 0.007936508 | IPI00015688.1 |
| GPD2 | 0.007936508 | IPI00017895.2 |
| GSPT1 | 0.015873016 | IPI00218829.9, IPI00909083.1 |
| GSTM4 | 0.031746032 | IPI00008770.1 |
| GYG1 | 0.015873016 | IPI00180386.5 |
| HARS2 | 0.015873016 | IPI00027445.1 |
| HEATR1 | 0.031746032 | IPI00024279.4 |
| HNRNPF | 0.031746032 | IPI00003881.5 |
| HSD17B12 | 0.015873016 | IPI00007676.3 |
| HSPA9 | 0.007936508 | IPI00007765.5 |
| HSPB1 | 0.007936508 | IPI00025512.2 |
| HSPE1 | 0.031746032 | IPI00220362.5 |
| IARS2 | 0.007936508 | IPI00017283.2 |
| ICAM1 | 0.015873016 | IPI00008494.4 |
| IGF2R | 0.015873016 | IPI00289819.4 |
| JUP | 0.015873016 | IPI00554711.3 |
| KIAA0368 | 0.007936508 | IPI00157790.7 |
| KPNA1 | 0.007936508 | IPI00303292.1 |
| KPNB1 | 0.015873016 | IPI00001639.2 |
| KRT13 | 0.007936508 | IPI00009866.6 |
| KRT14 | 0.015873016 | IPI00384444.5 |
| KRT15 | 0.007936508 | IPI00290077.2 |
| KRT16 | 0.007936508 | IPI00217963.3 |
| KRT17 | 0.007936508 | IPI00450768.7 |
| KRT18 | 0.031746032 | IPI00554788.5 |
| KRT4 | 0.031746032 | IPI00290078.5 |
| KRT5 | 0.007936508 | IPI00009867.3 |
| KRT6A | 0.007936508 | IPI00300725.7 |
| KRT7 | 0.007936508 | IPI00306959.10, IPI00847342.1 |
| LMAN1 | 0.015873016 | IPI00026530.4 |
| LPCAT1 | 0.007936508 | IPI00171626.3 |
| LPP | 0.015873016 | IPI00023704.1 |
| LRPPRC | 0.007936508 | IPI00783271.1 |
| LSS | 0.015873016 | IPI00009747.1 |
| MARCKS | 0.007936508 | IPI00219301.7 |
| MARS | 0.031746032 | IPI00008240.2 |
| MCM6 | 0.031746032 | IPI00031517.1 |
| MDK | 0.015873016 | IPI00010333.1 |
| METTL1 | 0.015873016 | IPI00290184.4 |
| MGST1 | 0.007936508 | IPI00021805.1 |
| MIA3 | 0.015873016 | IPI00455473.2 |
| MRPS7 | 0.015873016 | IPI00006440.6 |
| NANS | 0.015873016 | IPI00147874.1 |
| NCBP1 | 0.015873016 | IPI00019380.1 |
| NDRG1 | 0.015873016 | IPI00022078.3 |
| NDUFS8 | 0.007936508 | IPI00010845.3 |
| NOL6 | 0.015873016 | IPI00152890.1 |
| NOMO3, NOMO1 | 0.031746032 | IPI00329352.3 |
| NOP5/NOP58 | 0.031746032 | IPI00006379.1 |
| NUDCD2 | 0.015873016 | IPI00103142.1 |
| NUP205 | 0.015873016 | IPI00783781.1 |
| OAS3 | 0.015873016 | IPI00002405.4 |
| PAICS | 0.015873016 | IPI00217223.1 |
| PAK2 | 0.007936508 | IPI00419979.3 |
| PDCD11 | 0.007936508 | IPI00400922.5 |
| PDCD5 | 0.031746032 | IPI00023640.3 |
| PFAS | 0.007936508 | IPI00004534.3 |
| PGM2L1 | 0.015873016 | IPI00173346.3 |
| PGRMC1 | 0.015873016 | IPI00220739.3 |
| PHGDH | 0.015873016 | IPI00011200.5 |
| PITRM1 | 0.031746032 | IPI00219613.4 |
| PKP1 | 0.007936508 | IPI00071509.1, IPI00218528.1 |
| PRDX3 | 0.031746032 | IPI00024919.3 |
| PRDX4 | 0.031746032 | IPI00011937.1 |
| PRRC1 | 0.015873016 | IPI00217053.6 |
| PSMA1 | 0.031746032 | IPI00016832.1 |
| PSMB2 | 0.015873016 | IPI00028006.1 |
| PSMB6 | 0.015873016 | IPI00000811.2 |
| PSMD13 | 0.007936508 | IPI00375380.4 |
| PSMD5 | 0.007936508 | IPI00002134.4 |
| PSMD6 | 0.015873016 | IPI00014151.3 |
| PYCR1 | 0.031746032 | IPI00376503.2, IPI00550882.3 |
| RAB3GAP1 | 0.015873016 | IPI00014235.3 |
| RARS | 0.007936508 | IPI00004860.2 |
| RNF213 | 0.015873016 | IPI00828098.1 |
| RNH1 | 0.031746032 | IPI00550069.3 |
| RPL17, LOC100133931 | 0.007936508 | IPI00413324.6 |
| RPL18 | 0.031746032 | IPI00215719.6 |
| RPLP1 | 0.031746032 | IPI00008527.3 |
| RPS3 | 0.015873016 | IPI00011253.3 |
| RSL1D1 | 0.031746032 | IPI00008708.5 |
| S100A10 | 0.007936508 | IPI00183695.9 |
| S100A13 | 0.007936508 | IPI00016179.1 |
| S100A2 | 0.015873016 | IPI00019869.3 |
| SAMM50 | 0.015873016 | IPI00412713.4 |
| SDF2L1 | 0.007936508 | IPI00106642.4 |
| SDHA | 0.007936508 | IPI00217143.3, IPI00305166.2 |
| SEC23IP | 0.015873016 | IPI00026969.4 |
| SEC24D | 0.015873016 | IPI00218288.6 |
| SEC31A | 0.015873016 | IPI00305152.6 |
| SERPINB5 | 0.015873016 | IPI00644196.1, IPI00783625.1 |
| SFN | 0.007936508 | IPI00013890.2 |
| SLC30A7 | 0.015873016 | IPI00302605.3 |
| SMS | 0.015873016 | IPI00005102.3 |
| SPRR1A | 0.031746032 | IPI00017987.2, IPI00914840.1 |
| SPRR1B | 0.007936508 | IPI00304903.4, IPI00873761.1 |
| SPRR3 | 0.007936508 | IPI00082931.1 |
| SRM | 0.015873016 | IPI00292020.3 |
| STAT6 | 0.015873016 | IPI00030782.1 |
| SYNJ2BP | 0.015873016 | IPI00299193.1 |
| TBCD | 0.015873016 | IPI00030774.2, IPI00396203.6 |
| TCP1 | 0.015873016 | IPI00290566.1 |
| TFRC | 0.015873016 | IPI00022462.2 |
| TJP2 | 0.031746032 | IPI00003843.1 |
| TMED7 | 0.031746032 | IPI00032825.2 |
| TMEM43 | 0.031746032 | IPI00301280.2 |
| TMOD3 | 0.015873016 | IPI00005087.1 |
| TPD52L2 | 0.031746032 | IPI00221178.1, IPI00306825.3 |
| TPP2 | 0.015873016 | IPI00020416.8 |
| TRAP1 | 0.007936508 | IPI00030275.5 |
| TRIM29 | 0.007936508 | IPI00073096.3 |
| TXNDC12 | 0.015873016 | IPI00026328.3 |
| UAP1 | 0.015873016 | IPI00000684.4 |
| UBA1 | 0.031746032 | IPI00645078.1 |
| UBR4 | 0.007936508 | IPI00180305.7, IPI00640981.3 |
| VASP | 0.031746032 | IPI00301058.5 |
| VAT1 | 0.007936508 | IPI00156689.3 |
| VDAC1 | 0.007936508 | IPI00216308.5 |
| VIM | 0.031746032 | IPI00418471.6 |
| WDR77 | 0.015873016 | IPI00012202.1 |

Table 4A and 4B. Proteins highly differentially expressed in NSCLC

TABLE 4B

| Name | ADC/SCC | SCC/ADC | p-value |
|---|---|---|---|
| TFRC | 0.10 | 10.2 | 0.016 |
| NDUFS8 | 0.08 | 12.3 | 0.008 |
| KRT14 | 0.07 | 13.7 | 0.016 |
| DSP | 0.07 | 14.4 | 0.008 |
| TRIM29 | 0.07 | 15.1 | 0.008 |
| GPC1 | 0.06 | 17.2 | 0.008 |
| SPRR1B | 0.06 | 17.6 | 0.008 |
| GSTM4 | 0.05 | 21.6 | 0.028 |
| DSC3 | 0.03 | 32.7 | 0.008 |
| SPRR1A | 0.03 | 33.3 | 0.028 |
| CALML3 | 0.03 | 33.9 | 0.008 |
| GBP6 | 0.02 | 43.8 | 0.028 |
| DSG3 | 0.02 | 44.9 | 0.008 |
| SPRR3 | 0.01 | 71.9 | 0.008 |
| ADH7 | 0.01 | 74.3 | 0.008 |
| PKP1 | 0.01 | 137 | 0.008 |
| KRT4 | 0.00 | 213 | 0.028 |
| CES1 | 0.00 | 284 | 0.008 |
| KRT16 | 0.00 | 461 | 0.008 |
| KRT15 | 0.00 | 483 | 0.008 |
| KRT5 | 0.00 | 667 | 0.008 |
| KRT6A | 0.00 | 756 | 0.008 |
| KRT13 | 0.00 | 1655 | 0.008 |

NB CPS1 5.02-fold ADC/SCC ratio, p = 0.027

TABLE 4A

| Name | ADC/SCC | SCC/ADC | p-value |
|---|---|---|---|
| RPS3 | 48.9 | 0.02 | 0.027 |
| GFPT1 | 46.8 | 0.02 | 0.012 |
| KPNB1 | 28.9 | 0.03 | 0.027 |
| KRT7 | 28.4 | 0.04 | 0.012 |
| EIF3B | 25.5 | 0.04 | 0.016 |
| LPCAT1 | 23.3 | 0.04 | 0.016 |
| C3 | 22.5 | 0.04 | 0.027 |
| GALE | 21.2 | 0.05 | 0.012 |
| CRABP2 | 21.1 | 0.05 | 0.027 |
| MGST1 | 19.5 | 0.05 | 0.012 |
| AP2A2 | 18.6 | 0.05 | 0.021 |
| AGR2 | 17.4 | 0.06 | 0.008 |
| ICAM1 | 17.1 | 0.06 | 0.027 |
| GORASP2 | 16.1 | 0.06 | 0.021 |
| GLRX | 15.5 | 0.06 | 0.012 |
| IARS2 | 15.1 | 0.07 | 0.008 |
| KIAA0368 | 14.3 | 0.07 | 0.012 |
| FKBP10 | 14.2 | 0.07 | 0.027 |
| S100A13 | 13.7 | 0.07 | 0.012 |
| CRIP2 | 12.3 | 0.08 | 0.021 |
| PGRMC1 | 12.1 | 0.08 | 0.027 |
| AIFM1 | 12.1 | 0.08 | 0.027 |
| SDF2L1 | 12.0 | 0.08 | 0.012 |
| GSPT1 | 11.2 | 0.09 | 0.027 |
| DNAJC10 | 11.0 | 0.09 | 0.021 |
| VIM | 10.3 | 0.10 | 0.036 |
| RNF213 | 10.3 | 0.10 | 0.027 |

This highly differential subset included 8 keratins, including 6 that were identified as 10-fold differentially expressed in the 1D data set. The proteins AGR2 and CPS1 were again identified as more abundant in ADC: 17.4-fold (p=0.008) for AGR2 (Table 4A), and 5.0-fold (p=0.03) for CPS1.

Of the 28 known human epithelial keratins (Moll et al. 2008), 22 were detected in the panel of xenografts, as summarized in Table 5.

TABLE 5
Keratin signatures in NSCLC.

| | | | Keratin type | | | | | | | | | | | |
|---|---|---|---|---|---|---|---|---|---|---|---|---|---|---|
| | | | ADC1 | | ADC2 | | ADC3 | | ADC4 | | ADC5 | | SCC1 | |
| | Protein | Type | AVG | CV | AVG | CV | AVG | CV | AVG | CV | AVG | CV | AVG | CV |
| Simple | KRT8 | II | 93.2 | 18% | 18.7 | 13% | 64.8 | 43% | 130.6 | 7% | 54.6 | 11% | 82.7 | 4% |
| Epithelial | KRT18 | I | 83.5 | 11% | 20.4 | 20% | 69.4 | 38% | 108.9 | 3% | 113.5 | 18% | 22.5 | 6% |
| | KRT20 | I | 4.9 | 45% | — | — | — | — | — | — | — | — | — | — |
| | KRT7 | II | 87.9 | 12% | 48.3 | 24% | 29.9 | 46% | 52.2 | 3% | 80.6 | 12% | — | — |
| | KRT19 | I | 92.3 | 11% | 4.7 | 16% | 18.4 | 35% | 115.6 | 26% | 54.5 | 30% | 295.0 | 8% |
| Stratified | KRT5 | II | — | — | — | — | — | — | — | — | — | — | 155.9 | 2% |
| Epethelial | KRT14 | I | — | — | 73.5 | 48% | — | — | — | — | — | — | 148.4 | 4% |
| | KRT15 | I | — | — | — | — | — | — | — | — | — | — | 183.1 | 3% |
| | KRT6A | II | — | — | — | — | — | — | — | — | — | — | 160.1 | 4% |
| | KRT6B | II | 7.6 | 7% | — | — | — | — | — | — | 6.5 | 13% | — | — |
| | KRT6C | II | — | — | — | — | — | — | — | — | — | — | 145.6 | 3% |
| | KRT16 | I | — | — | — | — | — | — | — | — | — | — | 118.0 | 3% |
| | KRT17 | I | 53.1 | 10% | 26.9 | 51% | 4.0 | 42% | 0.3 | 0% | 23.5 | 13% | 55.6 | 2% |
| | KRT4 | II | — | — | — | — | — | — | — | — | — | — | 184.1 | 5% |
| | KRT13 | I | — | — | — | — | — | — | — | — | — | — | 505.4 | 5% |
| | KRT1 | II | — | — | — | — | — | — | — | — | — | — | — | — |
| | KRT10 | I | — | — | — | — | — | — | — | — | — | — | 8.6 | 27% |
| | KRT2 | II | — | — | — | — | — | — | — | — | — | — | — | — |
| | KRT3 | II | — | — | — | — | — | — | — | — | — | — | — | — |
| | KRT76 | II | — | — | — | — | — | — | — | — | — | — | 38.4 | 4% |
| | KRT78 | II | — | — | — | — | — | — | — | — | — | — | 6.7 | 7% |
| | KRT80 | II | — | — | — | — | — | — | — | — | — | — | 38.1 | 2% |

| | | | Keratin type | | | | | | | | | | |
|---|---|---|---|---|---|---|---|---|---|---|---|---|---|
| | | | SCC2 | | SCC3 | | SCC4 | | SCC5 | | ADC/ | SCC/ | Wilcoxon |
| | Protein | Type | AVG | CV | AVG | CV | AVG | CV | AVG | CV | SC | ADC | p-value |
| Simple | KRT8 | II | 22.9 | 2% | 45.1 | 8% | 31.1 | 35% | 32.8 | 7% | 1.7 | 0.6 | 0.310 |
| Epithelial | KRT18 | I | 5.9 | 16% | 33.7 | 6% | 10.3 | 26% | 18.6 | 13% | 4.3 | 0.2 | 0.032 |
| | KRT20 | I | — | — | — | — | — | — | — | — | 5.9 | — | — |
| | KRT7 | II | — | — | 9.8 | 19% | — | — | — | — | 28.4 | 0.04 | 0.008 |
| | KRT19 | I | 57.0 | 9% | 61.3 | 2% | 196.2 | 26% | 94.9 | 9% | 0.4 | 2.5 | 0.151 |
| Stratified | KRT5 | II | 179.0 | 7% | 101.1 | 6% | 190.7 | 23% | 98.9 | 7% | — | 667.3 | 0.008 |
| Epethelial | KRT14 | I | 457.4 | 7% | 61.2 | 39% | 110.0 | 18% | 243.0 | 29% | 0.1 | 13.7 | 0.016 |
| | KRT15 | I | 114.9 | 3% | 41.1 | 6% | 96.1 | 21% | 90.4 | 34% | — | 483.3 | 0.008 |
| | KRT6A | II | 238.9 | 2% | 88.6 | 4% | 179.6 | 27% | 154.7 | 0.3% | — | 755.8 | 0.008 |
| | KRT6B | II | — | — | — | — | 166.4 | 30% | — | — | 0.1 | 11.3 | 1.000 |
| | KRT6C | II | 215.2 | 3% | — | — | — | — | — | — | — | 332.2 | 0.690 |
| | KRT16 | I | 146.3 | 2% | 13.5 | 12% | 84.2 | 30% | 139.6 | 46% | — | 461.2 | 0.008 |
| | KRT17 | I | 167.0 | 6% | 121.7 | 7% | 96.0 | 22% | 134.3 | 57% | 0.2 | 5.3 | 0.008 |
| | KRT4 | II | 18.3 | 27% | 0.2 | 0% | 20.9 | 18% | 8.2 | 64% | — | 213.0 | 0.032 |
| | KRT13 | I | 507.7 | 3% | 139.8 | 1% | 294.1 | 18% | 352.5 | 37% | — | 1654.8 | 0.008 |
| | KRT1 | II | 5.2 | 16% | — | — | 9.8 | 48% | — | — | — | 14.3 | 0.421 |
| | KRT10 | I | — | — | — | — | 13.5 | 28% | — | — | — | 20.9 | 0.421 |
| | KRT2 | II | — | — | — | — | 71.0 | 35% | — | — | — | 66.1 | — |
| | KRT3 | II | — | — | — | — | 52.2 | 27% | — | — | — | 48.7 | — |
| | KRT76 | II | 58.0 | 6% | 0.2 | 0% | 54.0 | 27% | — | — | — | 138.7 | 0.151 |
| | KRT78 | II | — | — | — | — | — | — | — | — | — | 6.8 | N/A |
| | KRT80 | II | — | — | — | — | — | — | — | — | — | 35.7 | N/A |

Ten KRT proteins were significantly differentially expressed between ADC and SCC subtypes (Table 5, boldface, KRTs 18, 7, 5, 14, 15, 6A, 16, 17, 4, 13). Table 5 is organized by grouping the KRT proteins according to their known expression in simple and stratified epithelia, and, where known, in type I/II pairs, which assemble as obligate heterodimers for intermediate filament assembly ^{5, 22.}

### Validation of Expression of Keratins and EGFR in NSCLC

In order to validate and extend the information on these proteins that was generated by 1D and 2D tandem MS, additional analyses were performed. This included IHC on FFPE tissue sections, Western immuno blotting, and SRM-MS. Table 1 summarizes the IHC information related to the EGFR and certain keratins. Table 6 lists the peptides and corresponding transitions that were measured as part of a single, mulitplexed SRM (or MRM) method that was used to scan the xenografts.

TABLE 6
Transitions measured by Multiplexed SRM/MRM

| Protein/ | Parent | | | |
|---|---|---|---|---|
| Peptide | Ion | Fragment | CE | Ion |
| KRT7 | 721.90 | 657.4 | 28 | y6 |
| LPDIFEAQIAGLR | | 857.5 | 28 | y8 |
| (SEQ ID NO: 2) | | 1004.6 | 28 | y9 |
| KRT7 | 636.86 | 729.5 | 25 | y7 |
| SLDLDGIIAEVK | | 844.5 | 25 | y8 |
| (SEQ ID NO: 3) | | 1072.6 | 25 | y10 |
| KRT19 | 695.35 | 676.3 | 27 | y6 |
| AALEDTLAETEAR | | 890.5 | 27 | y8 |
| (SEQ ID NO: 4) | | 1005.5 | 27 | y9 |
| KRT19 | 677.81 | 748.3 | 26 | y6 |
| SQYEVMAEQNR | | 847.4 | 26 | y7 |
| (SEQ ID NO: 5) | | 1139.5 | 26 | y9 |
| KRT5 | 547.27 | 602.3 | 22 | y5 |
| AQYEEIANR | | 731.4 | 22 | y6 |
| (SEQ ID NO: 6) | | 894.4 | 22 | y7 |
| KRT5 | 556.29 | 610.3 | 22 | y6 |
| ISISTSGGSFR | | 711.3 | 22 | y7 |
| (SEQ ID NO: 7) | | 798.4 | 22 | y8 |
| KRT14 | 713.35 | 849.4 | 28 | y9 |
| APSTYGGGLSVSS | | 906.5 | 28 | y10 |
| SR | | 1069.5 | 28 | y11 |
| (SEQ ID NO: 8) | | | | |
| KRT15 | 911.45 | 1266.6 | 34 | y16 |
| GGSLLAGGGGFGG | | 1323.6 | 34 | y17 |
| GSLSGGGGSR | | 1394.6 | 34 | y18 |
| (SEQ ID NO: 9) | | | | |
| KRT15 | 688.31 | 811.4 | 27 | y9 |
| FVSSGSGGGYG | | 955.4 | 27 | y11 |
| GGMR | | 1129.5 | 27 | y13 |
| (SEQ ID NO: 10) | | | | |
| KRT13 | 624.85 | 715.4 | 25 | y6 |
| LKYENELALR | | 844.5 | 25 | y7 |
| (SEQ ID NO: 11) | | 1007.5 | 25 | y8 |
| EGFR | 625.35 | 402.2 | 25 | y4 |
| NLQEILHGAVR | | 539.3 | 25 | y5 |
| (SEQ ID NO: 12) | | 765.5 | 25 | y7 |
| | | 894.5 | 25 | y8 |
| | | 1022.6 | 25 | y9 |
| EGFR | 604.87 | 529.3 | 24 | y4 |
| IPLENLQIIR | | | | y9 |
| (SEQ ID NO: 13) | | 548.3 | 24 | 2+ |
| | | 756.5 | 24 | y6 |
| | | 885.5 | 24 | y7 |
| | | 998.6 | 24 | y8 |
| GAPDH | 882.40 | 743.3 | 33 | y6 |
| LISWYDNEFGYS | | 1101.5 | 33 | y9 |
| NR | | 1264.5 | 33 | y10 |
| (SEQ ID NO: 14) | | | | |
| PKP1 | 711.38 | 946.5 | 28 | y9 |
| GLMSSGMSQLIG | | 1033.6 | 28 | y10 |
| LK | | 1120.6 | 28 | y11 |
| (SEQ ID NO: 15) | | | | |
| PKP1 | 659.35 | 617.3 | 26 | y6 |
| NMLGTLAGANSL | | 959.5 | 26 | y10 |
| R | | 1072.6 | 26 | y11 |
| (SEQ ID NO: 16) | | | | |

### Keratins

In FIG. 5A (and summarized in Table 1), IHC verified the expression of KRT7 in ADC samples, as well as low-level expression in SCC3. MS analysis by spectral counting and SRM provided quantitative results that were consistent with each other, and the IHC staining pattern (FIG. 5B). The spectral counting analysis detected KRT7 in SCC3 to a greater extent than SRM. The SRM data were reproducible, and results from two distinct KRT7 peptides (detailed in Table 6) were very similar. All keratin peptides subjected to SRM analysis were uniquely human, and therefore not subject to interference from murine orthologs.

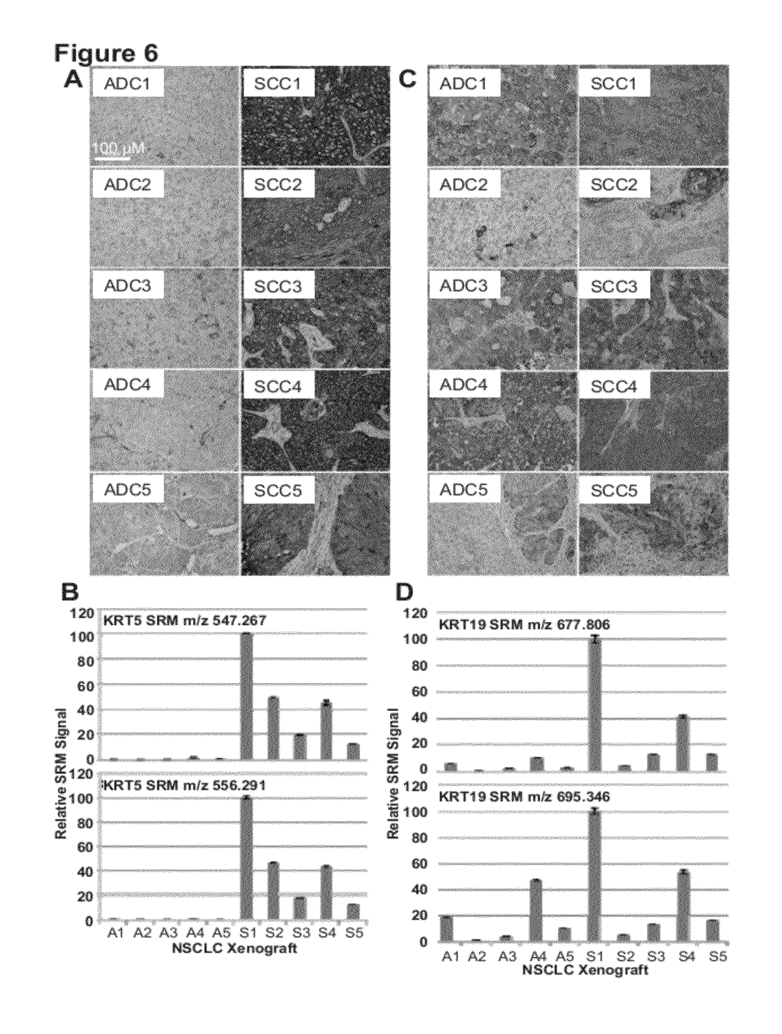

The IHC staining for keratins 5 and 6 (CK5/6) was negative for the ADC samples, and 100% positive for the SCC xenografts (Table 1, FIG. 6A). This is consistent with the unique expression in SCC compared with ADC measured by 2D LC-MS/MS (Table 5). SRM was used to measure two distinct KRT5 peptides (Table 6). The results of 4 SRM measurements (2 technical replicates for 2 peptides), were almost superimposable (FIG. 6B), indicating an accurate measure of KRT5, and showing a greater dynamic range than IHC.

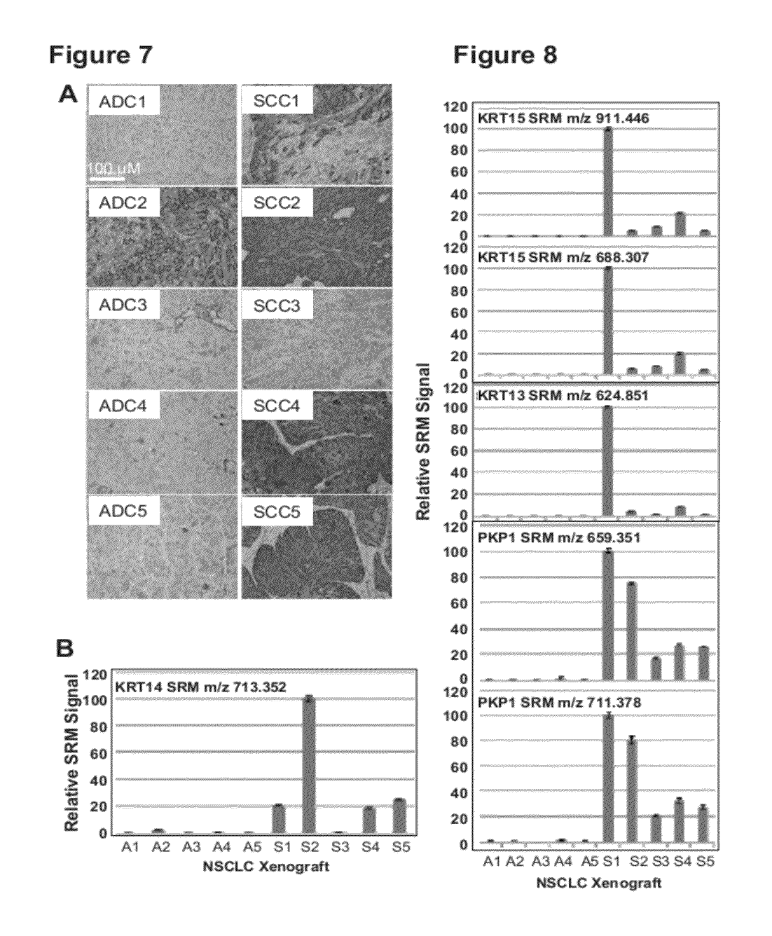

Analysis of KRT19 by IHC and SRM was similar: Replicate measurement of two KRT19 peptides (Table 6) gave very similar results (FIG. 6D), which were in general agreement with the spectral counting data (Table 5), and IHC, but with apparently greater dynamic range than IHC staining (FIG. 6C, Table 1). A similar trend was observed for KRT14 wherein the SRM measurements (FIG. 7B) were similar to spectral counting (Table 5), including the maximal signal seen in SCC2 and low-level signals in ADC2 and SCC3. This was generally consistent with the IHC results (Table 1, FIG. 7A), except that SCC3 was scored negative by IHC (Table 1, FIG. 7A).

FIG. 8 provides additional SRM measurements that were simultaneously collected as part of the multiplexed method. KRT15 was measured by following the transitions of two distinct human peptides, and gave near identical results with minimal variance. This indicated SCC-specific expression of KRT15, consistent with spectral counting (Table 5). KRT13 was measured as a function of 3 transitions from a single peptide ion. Plakophilin-1 (PKP1) was also observed to show a distinctive SCC-positive, ADC-negative expression pattern by spectral counting, and this was confirmed by SRM measurement of two peptides that gave near identical results.

### The EGF Receptor

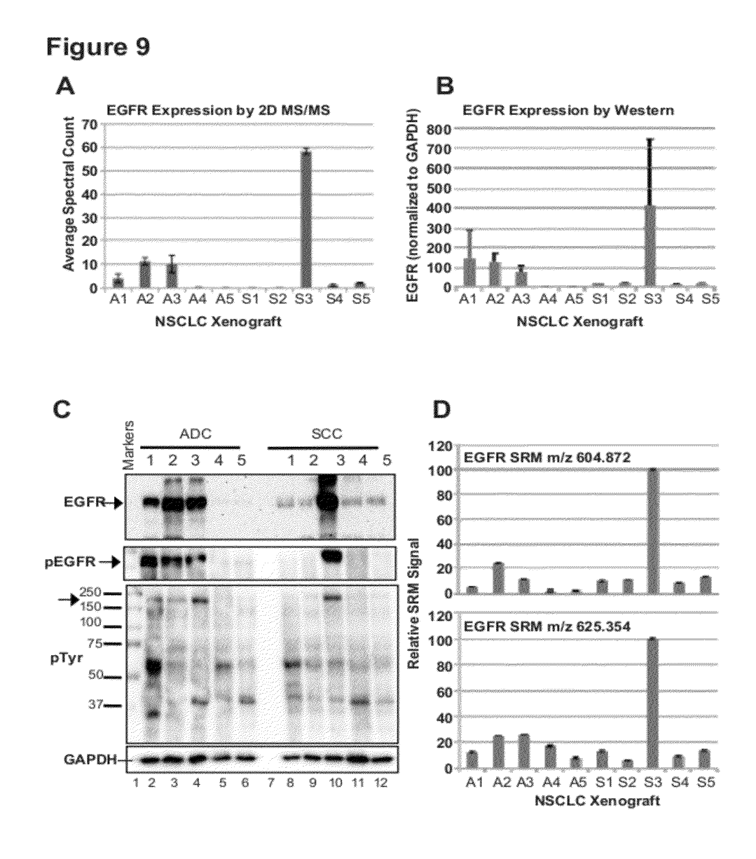

By 2D LC-MS/MS analysis the EGFR was identified in 6 xenografts, 3-each ADC and SCC, but was not identified as differentially expressed between the two subtypes. To further examine EGFR expression and activation, additional data were generated by application of IHC (Table 1), Western immuno blotting, and SRM-MS. As shown in FIG. 9, results obtained by MS/MS spectral counting (FIG. 9A), Western blotting (FIG. 9B, 9C) and SRM analysis of two different EGFR peptides (FIG. 9D) were similar in their identification of SCC3 as having the relative highest EGFR expression level. Quantification of triplicate Western blot chemiluminescence was associated considerable variation, and apparently limited dynamic range compared to the MS methods, but was sensitive in its apparent detection of EGFR in samples SCC1 and SCC2, which was not detected by spectral counting. The SRM measurements were sufficiently sensitive to detect EGFR in all 10 samples. The SRM measurements were made in duplicate, and the results were associated with very minimal variation (FIG. 9D). Moreover, a very similar pattern of expression was obtained by monitoring transitions associated with the two different EGFR peptides. The EGFR peptide m/z 604.87 is identical in murine and human species, whereas the peptide m/z 625.35 is distinctively human (Table 6).

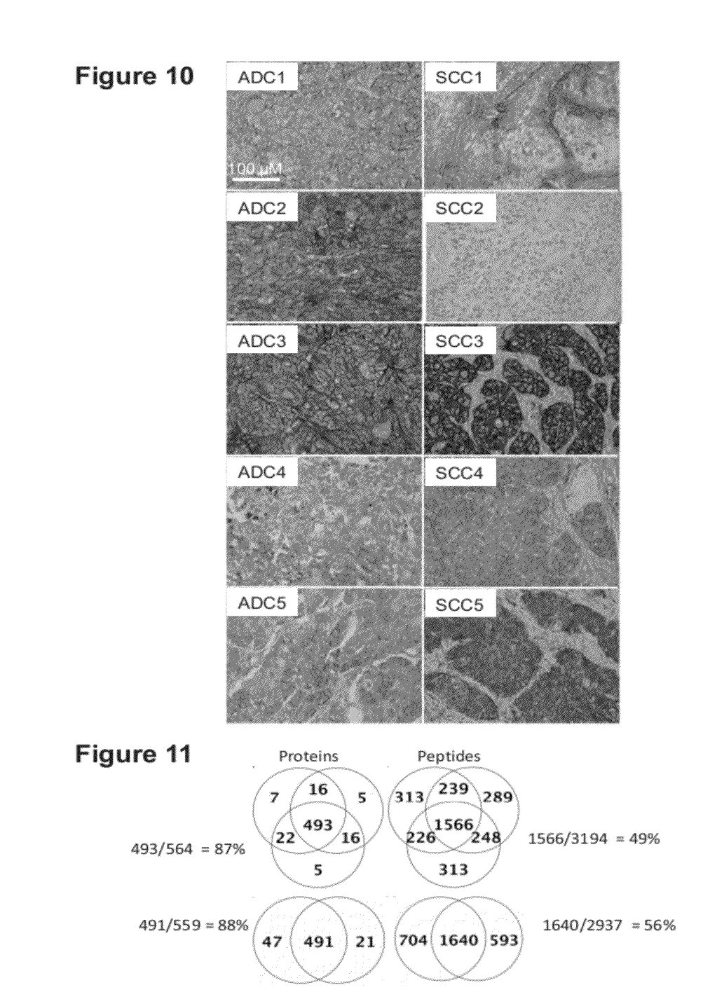

Phosphotyrosine (pTyr or pY)-directed Western blotting was used to assess the activation of EGFR in the xenografts. Tyr¹⁰⁶⁸ becomes phosphorylated upon EGFR activation, and through direct binding of the adaptor protein GRB2 is coupled to stimulation of the RAS-ERK signaling axis ²³. Probing with antibodies to pY¹⁰⁶⁸ provided qualitative results indicating activation of EGFR to some extent in ADCs 1-3 and SCC3. Activation in ADC3 was expected since this xenograft harbors the EGFR kinase-domain-activating exon 19 deletion (Table 1). Staining of whole-tissue extracts with antibodies to pTyr revealed a prominent band at the expected size of the EGFR in ADC3 and SCC3, and to a lesser extent in ADCs 1 and 2. This is consistent the pY¹⁰⁶⁸ staining, and suggests EGFR was activated in SCC3 in addition to its relatively high level of expression. The anti-pTyr blot also indicated distinct patterns of pY-proteins in the tumors. A strong signal migrating at Mr 55K-65K was evident in ADC1. Discernible bands at Mr 62K were present in ADCs 2 and 4, and SCCs 1-3, and bands at approximately Mr 38K in ADC3, ADC5, SCC4. Tissue micro array and subjective IHC scoring (Table 1) were in general agreement with the EGFR expression data presented in FIG. 9, but did not highlight the relatively high level of EGFR in SCC3. The IHC fields shown in FIG. 10 are consistent with highest EGFR expression in SCC3, and generally reflect well the profile of EGFR expression seen by SRM.

### DISCUSSION

### The Strategic Application of Proteomics for Tumor Profiling

The purpose of this pilot study was twofold. First, to determine the feasibility of using a proteomics platform comprised of a high resolution LC-MS/MS instrument for 1D and 2D comprehensive protein signature discovery, and combined with an LC-triple quadrupole instrument for multiplexed SRM-based relative quantification of signature proteins of interest. A similar approach of protein profiling leading to SRM/MRM-based quantification was effectively applied as part of a comprehensive platform to characterize a mouse model of breast cancer ²⁴. The second goal was to glean insights into the protein profiles expressed in a perceived information-rich resource represented by xenografts established from primary resected tumors. Proteomic profiles lacking detailed protein identifications have been shown to effectively stratify NSCLC tumors ²⁵. This report provides the most detailed analysis of protein expression in NSCLC to-date, and demonstrated effective recognition of ADC and SCC subtypes based on their unique proteomics signatures. The set of 10 tumors used in this pilot study did not include examples of the more rare, large cell carcinoma. It is expected that as data accumulate by analysis of a greater diversity of xenografts, it is likely that the proteomics profiles will stratify into more groups than the traditionally recognized ADC, SCC and large cell subtypes. The effective translation of proteomic signatures into multiplexed SRM (or MRM) assays, which may be applied to quantify proteins in minute surgical samples, represents a new strategy to stratify tumors. This will facilitate, in the first instance, case controlled studies of outcome that may correlate with an expanded set of proteome-defined tumor subtypes.

The quantitative results from 2D LC-MS/MS spectral counting and SRM were remarkably consistent with each other, and complemented the IHC observations. Compared to the 2D method, the 1D LC-MS/MS approach was simpler and faster, both technically and with respect to data analysis. It revealed the highly differential expression of both several keratins and the two urea cycle enzymes. The 2D findings verified the results of the 1D analysis and provided a more statistically robust data set, and with greater proteome coverage. Both approaches resulted in the identification of a set of highly differentially expressed proteins that were detected at levels differing by at least 10-fold between the 5 ADC and 5 SCC tumor-derived xenografts. More than 200 proteins (216) were identified as statistically different between ADC and SCC tumors, which includes 178 identified the 2D data set and another non-overlapping 38 from the 1D analysis.

A key element of the experimental plan was the comparison of traditional pathology laboratory methods such as IHC with quantitative proteomics. The MS-based proteomics results were verified by the IHC and Western data. In terms of tumor characterization, the SRM results complemented IHC which retains the advantage of revealing protein subcellular localization and cellular organization and heterogeneity.

### Keratin Structure and Function and Clinical Relevance

Among the most abundant proteins detected by MS analysis were epithelial keratins, and they were among the most strikingly highly differentially expressed. For example, six KRTs (5, 15, 6A, 16, 4, 13) were detected exclusively in SCC (Table 5). As expected, the ADC xenografts were predominantly characterized by keratins typically associated with simple epithelia; SCC also expressed these to some extent, but were notable for their expression of KRTs associated with stratified epithelium. Some but not all ADC also expressed very low levels of the squamous-type (i.e. stratified epithelial) keratins

KRT14 and KRT17. The less characterized KRT80, which was detected previously in lung ²⁶ was identified in one SCC tumor (SCC1), and three SCC xenografts expressed the rare KRT78 (refer to Table 5). SCC3 was unique both in its high level expression of EGFR, and as the only SCC found to express KRT7, which was otherwise only seen in ADC. The KRT7 expression in this instance may be an indication SCC3 arose through squamous metaplasia. The keratins are a key structural component of the 3-dimensional epithelial barrier ⁵. In reference to the role of keratins in epithelia, Moll et al. ⁵ stated “this main cytoskeletal function transcends the single cell level.” Hence, the measurement of KRT proteins in the primary xenograft model provides insight into a key structural component of three dimensional lung tumors. van Dorst et al. ²⁷ examined by IHC a limited set of keratins in adenocarcinomas and squamous cell carcinomas, including 16 from lung, and noted the difficulty in classifying the tumors by this method. The ability to comprehensively measure KRTs as demonstrated in this study suggests that efficient classification of ADC and SCC subtypes may be achieved, if not assisted by multiplexed-SRM-mediated, comprehensive KRT profiles.

Plakophilin-1 (PKP1) was also found highly differentially expressed in SCC, and functions in the linkage of intermediate filaments to desmosomes²⁸. Interestingly, PKP1 over expression correlates with increased cell proliferation and size, and regulates translation through interaction with eIF4A1²⁹. The related protein PKP3 is up-regulated and oncogenic in NSCLC ³⁰. While the present study was not aimed at identifying target proteins differentially expressed between tumor and normal tissue, this example illustrates the ability to discover and link tumor molecular markers with the cancer phenotype. It is predicted that the biomarkers will be differentially expressed between tumor and normal tissue. [The discovery of differential expression of the urea cycle enzyme CPS1 illustrates the potential monitoring of metabolic profiles by proteomics. Elevated ARG2 in NSCLC, and in large cell carcinoma in particular, was observed previously ³¹. Mutationally-activated forms of EGFR are recognized drug targets in NSCLC, but how EGFR markers, such as EGFR protein expression, gene copy number, and mutation status, should be incorporated into clinical decision making remains an evolving and contentious issue ³². EGFR expression was expected in both ADC and SCC, and was reproducibly relatively quantified by spectral counting and SRM. These results complement well the measurement of EGFR by IHC, which informs of positive cells, and subcellular localization (e.g. peripheral staining corresponding to plasma membrane localization). However, it has been recognized that various EGFR mutations, ligands, and therapeutic treatments can differentially affect EGFR protein stability and subcellular localization. Therefore, the accurate quantification of EGFR protein levels by SRM may enable the further stratification of NSCLC in terms of EGFR levels, beyond what has been achieved by IHC, measures of gene copy number, and mutations. In the 10 xenografts analyzed in this study, the range in EGFR expression was approximately 50-fold. Also, SRM measurement of a spiked-in, stable-isotope-labeled EGFR peptide (identical in sequence to the m/z 604.87 peptide, Table 6) allowed estimation of the level of EGFR at approximately 6×10⁵ copies per cell in SCC3, which expressed the highest amount of EGFR. These examples illustrate the potential application of SRM to quantify drug target protein levels with greater precision than is achieved by current methods such as IHC. This may facilitate a better assessment of correlations in EGFR protein levels and responsiveness to EGFR-directed drugs.

The sensitivity and versatility (i.e. multiplexing) of SRM enabled the assembly of a single assay to measure keratins, the target EGFR, and examples of metabolic enzymes. A more detailed examination of human/tumor and murine/stroma material is under investigation. In conclusion, the methods and compositions are useful for the development of SRM-based assays to measure NSCLC subtypes, and the levels of expression and activation of validated drug targets such as the EGFR (and phosphorylated EGFR), and metabolic enzyme. Additional clinical utility will be realized as these methods are further adapted for the analysis of FFPE patient tissue specimens, as recently demonstrated ³³. This information and strategic approach has the potential improve the recognition and treatment of NSCLC, and other cancers.

While the present disclosure has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. All sequences (e.g. nucleotide, including RNA and cDNA, and polypeptide sequences) of genes listed in Table 2, 4A, 4B, 6 and/or 7, for example referred to by accession number are herein incorporated specifically by reference.

### REFERENCES

1. Rosell, R.; Felip, E.; Garcia-Campelo, R.; Balana, C., The biology of non-small-cell lung cancer: identifying new targets for rational therapy. Lung Cancer 2004, 46, (2), 135-48.
2. Scagliotti, G.; Hanna, N.; Fossella, F.; Sugarman, K.; Blatter, J.; Peterson, P.; Simms, L.; Shepherd, F. A., The differential efficacy of pemetrexed according to NSCLC histology: a review of two Phase III studies. Oncologist 2009, 14, (3), 253-63.
3. Coate, L. E.; John, T.; Tsao, M. S.; Shepherd, F. A., Molecular predictive and prognostic markers in non-small-cell lung cancer. Lancet Oncol 2009, 10, (10), 1001-10.
4. Tsao, M. S.; Sakurada, A.; Cutz, J. C.; Zhu, C. Q.; Kamel-Reid, S.; Squire, J.; Lorimer, I.; Zhang, T.; Liu, N.; Daneshmand, M.; Marrano, P.; da Cunha Santos, G.; Lagarde, A.; Richardson, F.; Sey mour, L.; Whitehead, M.; Ding, K.; Pater, J.; Shepherd, F. A., Erlotinib in lung cancer—molecular and clinical predictors of outcome. N Engl J Med 2005, 353, (2), 133-44.
5. Moll, R.; Divo, M.; Langbein, L., The human keratins: biology and pathology. Histochem Cell Biol 2008, 129, (6), 705-33.
6. Chen, F.; Luo, X.; Zhang, J.; Lu, Y.; Luo, R., Elevated serum levels of TPS and CYFRA 21-1 predict poor prognosis in advanced non-small-cell lung cancer patients treated with gefitinib. Med Oncol 2009.
7. de Hoog, C. L.; Mann, M., Proteomics. Annu Rev Genomics Hum Genet 2004, 5, 267-93.
8. Troiani, T.; Schettino, C.; Martinelli, E.; Morgillo, F.; Tortora, G.; Ciardiello, F., The use of xenograft models for the selection of cancer treatments with the EGFR as an example. Crit Rev Oncol Hematol 2008, 65, (3), 200-11.
9. Lange, V.; Picotti, P.; Domon, B.; Aebersold, R., Selected reaction monitoring for quantitative proteomics: a tutorial. Mol Syst Biol 2008, 4, 222.
10. Tong, J.; Taylor, P.; Jovceva, E.; St-Germain, J.; Jin, L.; Nikolic, A.; Gu, X.; Li, Z., Trudel, S; Moran, M., Tandem Immunoprecipitation of phosphotyrosine-mass spectrometry (TIPY-MS) indicates C19orf19 becomes tyrosine phosphorylated and associated with activated epidermal growth factor receptor. J. Proteome Res. 2008, 7, (3), 1067-77.
11. St-Germain, J. R.; Taylor, P.; Tong, J.; Jin, L. L.; Nikolic, A.; Stewart, I. I.; Ewing, R. M.; Dharsee, M.; Li, Z. H.; Trudel, S.; Moran, M. F., Multiple Myeloma Phosphotyrosine Proteomic Profile Associated with FGFR3 expression, ligand activation, and drug inhibition. Proc Natl Acad Sci, USA 2009, 106, (47), 20127-20132.
12. Taylor, P.; Nielsen, P. A.; Trelle, M. B.; Horning, O. B.; Andersen, M. B.; Vorm, O.; Moran, M. F.; Kislinger, T., Automated 2D peptide separation on a 1D nano-LC-MS system. J Proteome Res 2009, 8, (3), 1610-6.
13. Cox, B.; Kotlyar, M.; Evangelou, A. I.; Ignatchenko, V.; Ignatchenko, A.; Whiteley, K.; Jurisica, I.; Adamson, S. L.; Rossant, J.; Kislinger, T., Comparative systems biology of human and mouse as a tool to guide the modeling of human placental pathology. Mol Syst Biol 2009, 5, 279.
14. Sodek, K. L.; Evangelou, A. I.; Ignatchenko, A.; Agochiya, M.; Brown, T. J.; Ringuette, M. J.; Jurisica, I.; Kislinger, T., Identification of pathways associated with invasive behavior by ovarian cancer cells using multidimensional protein identification technology (MudPIT). Mol Biosyst 2008, 4, (7), 762-73.
15. Zybailov, B.; Coleman, M. K.; Florens, L.; Washburn, M. P., Correlation of relative abundance ratios derived from peptide ion chromatograms and spectrum counting for quantitative proteomic analysis using stable isotope labeling. Anal Chem 2005, 77, (19), 6218-24.
16. Zybailov, B.; Mosley, A. L.; Sardiu, M. E.; Coleman, M. K.; Florens, L.; Washburn, M. P., Statistical analysis of membrane proteome expression changes in Saccharomyces cerevisiae. J Proteome Res 2006, 5, (9), 2339-47.
17. Drake, R. R.; Elschenbroich, S.; Lopez-Perez, O.; Kim, Y.; Ignatchenko, V.; Ignatchenko, A.; Nyalwidhe, J. O.; Basu, G.; Wilkins, C. E.; Gjurich, B.; Lance, R. S.; Semmes, O. J.; Medin, J. A.; Kislinger, T., In-depth proteomic analyses of direct expressed prostatic secretions. J Proteome Res 2010, 9, (5), 2109-16.
18. Kislinger, T.; Rahman, K.; Radulovic, D.; Cox, B.; Rossant, J.; Emili, A., PRISM, a generic large scale proteomic investigation strategy for mammals. Mol Cell Proteomics 2003, 2, (2), 96-106.
19. Elschenbroich, S.; Ignatchenko, V.; Sharma, P.; Schmitt-Ulms, G.; Gramolini, A. O.; Kislinger, T., Peptide separations by on-line MudPIT compared to isoelectric focusing in an off-gel format: application to a membrane-enriched fraction from C2C12 mouse skeletal muscle cells. J Proteome Res 2009, 8, (10), 4860-9.
20. Washburn, M. P.; Wolters, D.; Yates, J. R., 3rd, Large-scale analysis of the yeast proteome by multidimensional protein identification technology. Nat Biotechnol 2001, 19, (3), 242-7.
21. Wolters, D. A.; Washburn, M. P.; Yates, J. R., 3rd, An automated multidimensional protein identification technology for shotgun proteomics. Anal Chem 2001, 73, (23), 5683-90.
22. Hatzfeld, M.; Franke, W. W., Pair formation and promiscuity of cytokeratins: formation in vitro of heterotypic complexes and intermediate-sized filaments by homologous and heterologous recombinations of purified polypeptides. J Cell Biol 1985, 101, (5 Pt 1), 1826-41.
23. Pawson, T., Specificity in signal transduction: from phosphotyrosine-SH2 domain interactions to complex cellular systems. Cell 2004, 116, (2), 191-203.
24. Whiteaker, J. R.; Zhang, H.; Zhao, L.; Wang, P.; Kelly-Spratt, K. S.; Ivey, R. G.; Piening, B. D.; Feng, L. C.; Kasarda, E.; Gurley, K. E.; Eng, J. K.; Chodosh, L. A.; Kemp, C. J.; McIntosh, M. W.; Paulovich, A. G., Integrated pipeline for mass spectrometry-based discovery and confirmation of biomarkers demonstrated in a mouse model of breast cancer. J Proteome Res 2007, 6, (10), 3962-75.
25. Yanagisawa, K.; Shyr, Y.; Xu, B. J.; Massion, P. P.; Larsen, P. H.; White, B. C.; Roberts, J. R.; Edgerton, M.; Gonzalez, A.; Nadaf, S.; Moore, J. H.; Caprioli, R. M.; Carbone, D. P., Proteomic patterns of tumour subsets in non-small-cell lung cancer. Lancet 2003, 362, (9382), 433-9.
26. Hesse, M.; Zimek, A.; Weber, K.; Magin, T. M., Comprehensive analysis of keratin gene clusters in humans and rodents. Eur J Cell Biol 2004, 83, (1), 19-26.
27. van Dorst, E. B.; van Muijen, G. N.; Litvinov, S. V.; Fleuren, G. J., The limited difference between keratin patterns of squamous cell carcinomas and adenocarcinomas is explicable by both cell lineage and state of differentiation of tumour cells. J Clin Pathol 1998, 51, (9), 679-84.
28. Bass-Zubek, A. E.; Godsel, L. M.; Delmar, M.; Green, K. J., Plakophilins: multifunctional scaffolds for adhesion and signaling. Curr Opin Cell Biol 2009, 21, (5), 708-16.
29. Wolf, A.; Krause-Gruszczynska, M.; Birkenmeier, O.; Ostareck-Lederer, A.; Huttelmaier, S.; Hatzfeld, M., Plakophilin 1 stimulates translation by promoting eIF4A1 activity. J Cell Biol 2010, 188, (4), 463-71.
30. Furukawa, C.; Daigo, Y.; Ishikawa, N.; Kato, T.; Ito, T.; Tsuchiya, E.; Sone, S.; Nakamura, Y., Plakophilin 3 oncogene as prognostic marker and therapeutic target for lung cancer. Cancer Res 2005, 65, (16), 7102-10.
31. Rotondo, R.; Mastracci, L.; Piazza, T.; Barisione, G.; Fabbi, M.; Cassanello, M.; Costa, R.; Morandi, B.; Astigiano, S.; Cesario, A.; Sormani, M. P.; Ferlazzo, G.; Grossi, F.; Ratto, G. B.; Ferrini, S.; Frumento, G., Arginase 2 is expressed by human lung cancer, but it neither induces immune suppression, nor affects disease progression. Int J Cancer 2008, 123, (5), 1108-16.
32. Shepherd, F. A.; Tsao, M. S., Epidermal growth factor receptor biomarkers in non-small-cell lung cancer: a riddle, wrapped in a mystery, inside an enigma. J Clin Oncol 2010, 28, (6), 903-5.
33. Taylor, P.; Tong, J.; Shih, W.; Darfler, M.; Tsao, M.; Krizman, D.; Eitner, C.; Moran, M., Detection and quantification of EGF receptor phosphorylation in formalin-fixed tumor sections by selected/multiple reaction monitoring mass spectrometry. Eur. J. Cancer 2009, 7, (4), 31.
34. Prakash, A.; Tomazela, D. M.; Frewen, B.; Maclean, B.; Merrihew, G.; Peterman, S.; Maccoss, M. J., Expediting the development of targeted SRM assays: using data from shotgun proteomics to automate method development. J Proteome Res 2009, 8, (6), 2733-9.

1. A method of screening for, diagnosing or detecting non-small cell lung carcinoma or an increased likelihood of developing non-small cell lung carcinoma in a subject comprising:
(a) determining a level of at least one biomarker associated with non-small cell lung carcinoma in a test sample from the subject, the at least one biomarker selected from the biomarkers set out in Table 2, 4A, 4B, 6 and/or 7; and
(b) comparing the level of the at least one biomarker in the test sample with a control;
wherein detecting a difference in a level of the at least one biomarker in the test sample compared to the control is indicative of whether the subject has or does not have non-small cell lung carcinoma or an increased likelihood of developing non-small cell lung carcinoma.

2. The method of claim 1, wherein the at least one biomarker associated with non-small cell lung carcinoma is selected from the biomarkers set out in Table 2, Table 4A and Table 4B.

3. The method of claim 1, wherein the difference in the level is an increase in the level of the at least one biomarker in the test sample compared to the control, wherein the increased level is indicative the subject has non-small cell lung carcinoma or an increased risk of developing non-small cell lung carcinoma.

4. The method of claim 1, wherein a ratio of the level of the at least one biomarker in the test sample compared to the control is greater than 2, 3, 5, 10, 12, 15, 20, 25, 30, 35, 40, 45, 50 or more.

5. The method of claim 1, wherein the non-small cell lung carcinoma is adenocarcinoma or squamous cell carcinoma.

6. The method of claim 1, comprising determining an expression profile in the test sample from the subject, the expression profile comprising a level for each of at least two biomarkers associated with non-small cell lung carcinoma, wherein the at least two biomarkers are selected from the biomarkers set out in Table 2, 4A, 4B, 6 and/or 7.

7. The method of claim 6, wherein the control is a reference profile associated with a non-small cell lung carcinoma subtype selected from adenocarcinoma and squamous cell carcinoma, and an expression profile most similar to the reference profile associated with adenocarcinoma is indicative that the subject has adenocarcinoma and an expression profile most similar to the reference profile associated with squamous cell carcinoma is indicative that the subject has squamous cell carcinoma.

8. The method of claim 1, wherein the at least one biomarker is a keratin, and the keratin is selected from KRT8, KRT18, KRT20, KRT7, KRT19, KRT5, KRT14, KRT15, KRT6A, KRT6B, KRT6C, KRT16, KRT17, KRT4, KRT13, KRT1, KRT10, KRT2, KRT3, KRT76, KRT78 and KRT80.

9. The method of claim 1, wherein an increased level of KRT5, KRT6 or KRT15 is indicative that the subject has non-small cell lung cancer of the squamous cell carcinoma subtype.

10. The method of claim 1, wherein an increased level of KRT7 is indicative that the subject has non-small cell lung cancer of the adenocarcinoma subtype or an increased level in CPS-1 and/or AGR2 is indicative that the subject has non-small cell lung cancer of the adenocarcinoma subtype.

11. The method of claim 1, wherein the biomarker is plakophilin-1 and an increased level is indicative that the subject has non-small cell lung cancer of the squamous cell carcinoma subtype.

12. A method according to claim 1 for differentiating between non-small cell lung carcinoma of the adenocarcinoma subtype and non-small cell lung carcinoma of the squamous cell carcinoma subtype in a subject, or detecting an increased likelihood of developing non-small cell lung carcinoma of the adenocarcinoma subtype or non-small cell lung carcinoma of the squamous cell carcinoma subtype in a subject comprising:
(a) determining a level of at least one biomarker associated with non-small cell lung carcinoma of the adenocarcinoma subtype or non-small cell lung carcinoma of the squamous cell subtype in a test sample from the subject wherein the at least one biomarker is selected from the biomarkers set out in Table 2, 4A, 4B, 6 and/or 7; and
(b) comparing the level of the at least one biomarker in the test sample with a control;
wherein detecting a difference or similarity in a level of the at least one biomarker in the test sample compared to the control is indicative of whether the subject has non-small cell lung carcinoma of the adenocarcinoma subtype or non-small cell lung carcinoma of the squamous cell carcinoma subtype, or an increased likelihood of developing non-small cell lung carcinoma of the adenocarcinoma subtype or non-small cell lung carcinoma of the squamous cell carcinoma subtype.

13. A method according to claim 1 for screening for, diagnosing or detecting non-small cell lung carcinoma of adenocarcinoma subtype or an increased likelihood of developing non-small cell lung carcinoma of the adenocarcinoma subtype in a subject comprising:
(a) determining a level of at least one biomarker associated with non-small cell lung carcinoma of the adenocarcinoma subtype in a test sample from the subject wherein the at least one biomarker is selected from the biomarkers set out in Table 2 or Table 4A; and
(b) comparing the level of the at least one biomarker in the test sample with a control;
wherein detecting a difference or similarity in the level of the at least one biomarker in the test sample compared to the control is indicative of the subject has or does not have non-small cell lung carcinoma of adenocarcinoma subtype or an increased likelihood of developing non-small cell lung carcinoma of adenocarcinoma subtype.

14. A method according to claim 1 for screening for, diagnosing or detecting non-small cell lung carcinoma of squamous cell carcinoma subtype or an increased likelihood of developing non-small cell lung carcinoma of squamous cell carcinoma subtype in a subject comprising:
(a) determining a level of at least one biomarker associated with non-small cell lung carcinoma of squamous cell carcinoma subtype in a test sample from the subject wherein the at least one biomarker is selected from the biomarkers set out in Table 2 or Table 4B; and
(b) comparing the level of the at least one biomarker in the test sample with a control;
wherein detecting a difference or similarity in the level of the at least one biomarker in the test sample compared to the control is indicative of whether the subject has or does not have non-small cell lung carcinoma of squamous cell carcinoma subtype or an increased likelihood of developing non-small cell lung carcinoma of squamous cell carcinoma subtype.

15. The method of claim 1, wherein the level of at least one biomarker is determined using mass spectrometry, wherein the mass spectrometry comprises tandem mass spectrometry, 1D LC MS/MS, 2D LC MS/MS, SRM and/or MRM.

16. The method of claim 15 wherein the biomarker is a biomarker listed in Table 6 and mass spectrometry is used to detect a peptide listed in Table 6.

17. A method of treating non-small cell lung carcinoma in a subject comprising:
a) diagnosing non-small cell lung carcinoma in a subject according to the method of claim 1; and
b) administering a treatment suitable for the treatment of non-small cell lung carcinoma to the subject.

18. A method of treating non-small cell lung carcinoma in a subject comprising:
a) diagnosing non-small cell lung carcinoma of adenocarcinoma subtype or non-small cell lung carcinoma of squamous cell carcinoma subtype in a subject according to the method of claim 12; and
b) administering to the subject a treatment suitable for treating non-small cell lung carcinoma of adenocarcinoma subtype when adenocarcinoma subtype is detected or administering a treatment suitable for treating non-small cell lung carcinoma of squamous cell carcinoma subtype when squamous cell carcinoma subtype is detected.

19. A SRM/MRM method for quantifying a level of at least one biomarker associated with non-small cell lung carcinoma in a sample, the method comprising the steps of:
a) isotope labeling a peptide fragment of the at least one biomarker wherein the at least one biomarker is selected from the biomarkers set out in Table 2, 4A, 4B, 6 and/or 7; and
b) evaluating the biomarker level using SRM/MRM mass spectrometry.

20. The method according to claim 19 wherein the SRM/MRM method comprises a dynamic detection range corresponding from about 10 000 copies to about 1 million copies of per cell.

21. The method according to claim 19, wherein the biomarker is the epidermal growth factor receptor, optionally phosphorylated epidermal growth factor receptor.

22. The method according to claim 21 wherein the peptide fragment is NLQEILHGAVR.

23. A method for treating non-small cell lung carcinoma in a subject comprising:
a) quantifying the level of EGFR in a test sample from the subject according to claim 19; and
b) administering an EGFR directed drug to the subject when the level of EGFR level quantified is above a threshold indicative that the subject would benefit from the EGFR directed drug.

24. A kit for measuring a level of at least one biomarker associated with non-small cell lung cancer or a subtype thereof in a sample, the at least one biomarker selected from the biomarkers set out in Table 2, 4A, 4B, 6 and/or 7, comprising:
a) a biomarker specific reagent, labeling isotope and/or a peptidase such as trypsin;
b) a kit control, optionally a peptide fragment of a biomarker;
c) optionally an array slide; and
d) optionally instructions for use.

25. The kit of claim 24, wherein the at least one biomarker is selected from the biomarkers set out in Table 2, Table 4A or 4B.


SEQUENCE LISTING (16 sequences)

SEQ ID NO: 1 - protein, 14 aa, Homo Sapien
LISWYDNEFGYSNR

SEQ ID NO: 2 - protein, 13 aa, Homo Sapien
LPDIFEAQIAGLR

SEQ ID NO: 3 - protein, 12 aa, Homo Sapien
SLDLDGIIAEVK

SEQ ID NO: 4 - protein, 13 aa, Homo Sapien
AALEDTLAETEAR

SEQ ID NO: 5 - protein, 11 aa, Homo Sapien
SQYEVMAEQNR

SEQ ID NO: 6 - protein, 9 aa, Homo Sapien
AQYEEIANR

SEQ ID NO: 7 - protein, 11 aa, Homo Sapien
ISISTSGGSFR

SEQ ID NO: 8 - protein, 15 aa, Homo Sapien
APSTYGGGLSVSSSR

SEQ ID NO: 9 - protein, 23 aa, Homo Sapien
GGSLLAGGGGFGGGSLSGGGGSR

SEQ ID NO: 10 - protein, 15 aa, Homo Sapien
FVSSGSGGGYGGGMR

SEQ ID NO: 11 - protein, 10 aa, Homo Sapien
LKYENELALR

SEQ ID NO: 12 - protein, 11 aa, Homo Sapien
NLQEILHGAVR

SEQ ID NO: 13 - protein, 10 aa, Homo Sapien
IPLENLQIIR

SEQ ID NO: 14 - protein, 14 aa, Homo Sapien
LISWYDNEFGYSNR

SEQ ID NO: 15 - protein, 14 aa, Homo Sapien
GLMSSGMSQLIGLK

SEQ ID NO: 16 - protein, 13 aa, Homo Sapien
NMLGTLAGANSLR